(19) United States
(12) Patent Application Publication
(10) Pub. No.: US 2019/0292310 A1
(43) Pub. Date: Sep. 26, 2019

(54) SYNTHESIS OF HIGH DENSITY MOLECULAR DNA BRUSHES VIA ORGANIC-PHASE RING-OPENING METATHESIS (CO)POLYMERIZATION

(71) Applicant: NORTHEASTERN UNIVERSITY, Boston, MA (US)

(72) Inventors: Ke ZHANG, Boston, MA (US); Xuyu TAN, Boston, MA (US)

(21) Appl. No.: 16/253,524

(22) Filed: Jan. 22, 2019

Related U.S. Application Data
(60) Provisional application No. 62/645,379, filed on Mar. 20, 2018.

(51) Int. Cl. C08G 61/08; C12N 15/113; C12Q 1/6886

(52) U.S. Cl. CPC C08G 61/08; C12N 15/113; C12Q 1/6886; C08G 2261/3324; C12N 2310/3535; C08G 2261/418; C12N 2310/3515; C12N 2310/51; C08G 2261/1432

(57) ABSTRACT

The invention provides novel synthetic methods for oligonucleotide polymerization reactions that separate the deprotection and cleavage step following solid-phase oligonucleotide synthesis into two separate steps, thereby providing fully protected hydrophobic oligonucleotides that can be further manipulated in organic solvents. The disclosed methods enable the synthesis of new structures, such as brush DNA and brush RNA polymers and micellar spherical nucleic acids (SNAs

### CROSS REFERENCE TO RELATED APPLICATIONS

This application claims the benefit of priority under 35 U.S.C. § 119 to U.S. Provisional Patent Application No. 62/645,379 by Zhang and Tan, filed Mar. 20, 2018, the entire disclosure of which is incorporated herein by reference thereto.

### STATEMENT OF GOVERNMENT INTERESTS

This invention was made with Government support under National Science Foundation Grant Number 14532555. The Government has certain rights in the invention.

### FIELD OF THE INVENTION

This application relates to a novel synthetic method for oligonucleotide polymerization reactions. The disclosed methods enable the synthesis of new structures, such as bottlebrush DNA or bottlebrush RNA polymers, and makes it easier to prepare structures such as pacDNA and pacRNA.

### BACKGROUND

Oligonucleotides have found extensive applications spanning structural DNA nanotechnology, materials assembly, DNA-encoded libraries, and DNA-based nanomedicine. In all of these fields it is often required for the oligonucleotide to be covalently attached to other moieties, be it a small molecule, a macromolecule, a crosslinked network, or a nanoscopic/macroscopic surface. These structures play irreplaceable roles in medicine, diagnostics, crystal engineering, and drug discovery. The success of existing structures underscores the importance of developing new reactions and methods that can covalently arrange nucleic acids into a wide variety of well-defined architectures. Examining the chemical structure of the DNA conjugates reported thus far, two observations can be made: (i) Conjugates with a single oligonucleotide can be synthesized by solid-phase coupling or solution-phase coupling and (ii) Conjugates with multiple oligonucleotides are made by solution-phase coupling almost exclusively. (Singh, Y., Murat, P. & Defrancq, E. Recent developments in oligonucleotide conjugation, Chem. Soc. Rev. 39, 2054-2070 (2010); Juliano, R. L., Ming, X. & Nakagawa, O. The chemistry and biology of oligonucleotide conjugates, Acc. Chem. Res. 45, 1067-1076 (2012)) These observations reveal an important limitation of the solid-phase methodology: architecturally complex structures involving multiple nucleic acid strands, such as DNA bottlebrushes and crosslinked networks, are difficult, if not impossible, to achieve on a rigid, 2-dimensional surface. Yet, nucleic acid-containing materials with a non-linear 3-dimensional architecture are an interesting class of structures that remain at the forefront of exploration in chemistry, materials science, and medicine. Solution-phase coupling provides only partial access to these materials. Amphiphilic conjugates, for example, are challenging by solution coupling due to the difficulty in finding a common solvent for the hydrophilic DNA and the hydrophobic segment. In addition, high-density, multivalent DNA constructs are difficult because of the strong repulsive interaction of negatively charged phosphate groups on the DNA backbone. (Hurst, S. J., Lytton-Jean, A. K. & Mirkin, C. A. Maximizing DNA loading on a range of gold nanoparticle sizes, Anal. Chem. 78, 8313-8318 (2006); Liu, B. & Liu, J. Methods for preparing DNA-functionalized gold nanoparticles, a key reagent of bioanalytical chemistry, Anal. Methods 9, 2633-2643 (2017); Sun, D. & Gang, O., DNA-functionalized quantum dots: fabrication, structural, and physicochemical properties. Langmuir 29, 7038-7046 (2013)).

Among the non-linear architectures, bottlebrush-type oligonucleotides have attracted considerable attention due to the increased local nucleic acid density, which has been suggested as a factor that facilitates increased cellular endocytosis and enables carrier-free cellular gene expression control. (Liu. B. and Liu, J., Methods for preparing DNA-functionalized gold nanoparticles, a key reagent of bioanalytical chemistry, Anal. Methods, 9, 2633-2643 (2017); Tan, X. et al., Blurring the role of oligonucleotides: spherical nucleic acids as a drug delivery vehicle, J. Am. Chem. Soc, 138, 10834-10837 (2016)) To circumvent the synthetic limitations, James et al. reported the organic-phase polymerization of peptide nucleic acids (PNAs) to prepare bottlebrushes, which takes advantage of the non-charged nature of PNAs and their solubility in dimethyl formamide (DMF). (James, C. R. et al., Poly (oligonucleotide). J. Am. Chem. Soc. 136, 11216-11219 (2014). Nonetheless, the method cannot be readily extended to natural nucleic acids with phosphodiester backbones. Liu et al. adopted cationic surfactants for electrostatic complexation with DNA as a more general means to neutralize the negative charge and improve lipophilicity. (Liu, K. et al. Nucleic acid chemistry in the organic phase: from functionalized oligonucleotides to DNA side chain polymers, J. Am. Chem. Soc. 136, 14255-14262 (2014)). Notwithstanding broader applicability, the reaction yields are moderate, especially for larger oligonucleotides/coupling partners and for polymerization. In addition, the complete removal of surfactants can be challenging. Thus, there is a need for an improved method of synthesizing oligonucleotides in the presence of organic solvents for polymerization reactions that eliminates the common limitations of prior methods of synthesis.

### SUMMARY OF THE INVENTION

In one aspect of the invention there is provided a method of synthesizing a protected norbornenyl DNA monomer or protected norbornenyl RNA monomer (protDNA) comprising the steps of:
1) providing a controlled pore glass (CPG) solid support for oligonucleotide synthesis, wherein the CPG solid support has covalently bound thereon a disulfide linker;
2) conducting a solid-phase oligonucleotide synthesis reaction with ultramild cyanoethyl (CE) phosphoramidites on the CPG solid support to form a protected oligonucleotide having a predetermined nucleotide sequence covalently bound at the 3′ end thereof to the disulfide linker;
3) cleaving the disulfide linker under conditions that release the protected oligonucleotide from the solid support, thereby forming a sulfhydryl group at the 3′end of the protected oligonucleotide; and
4) coupling an iodoacetyl- or a maleimide-functionalized norbornene monomer to the sulfhydryl group of the protected oligonucleotide via a SN₂ or Michael addition reaction, respectively, to produce a protected norbornenyl DNA monomer or protected norbornenyl RNA monomer.

In another aspect of the invention there is provided a method of synthesizing a DNA bottlebrush homopolymer comprising the steps of:
1) conducting ring opening metathesis polymerization (ROMP) of a plurality of protected norbornenyl DNA monomers in the presence of an organic solvent and a heavy metal catalyst at a monomer:catalyst ratio of from 2:1 to 200:1 to form a DNA bottlebrush comprising a desired nucleotide sequence;
2) removing the catalyst; and
3) treating the DNA bottlebrush with a deprotecting agent.

In another aspect of the invention there is provided a method of synthesizing pacDNA comprising
1) reacting amine-terminated noncationic biocompatible monomers selected with norbornenyl N-hydroxysuccinimide ester to provide norbornenyl noncationic monomers;
2) dissolving the norbornenyl noncationic monomers in an organic solvent to form a first solution;
3) dissolving a plurality of protected norbornenyl DNA monomer (protDNA) in an organic solvent to form a second solution;
4) forming a ROMP reaction mixture by adding a heavy metal catalyst to the first solution at a monomer:catalyst molar ratio of 10:1 to 100:1 and polymerizing the norbornenyl noncationic monomers in the ROMP reaction mixture to form a first block polymer having a predetermined sequence;
5) adding the second solution to the ROMP reaction mixture to provide a monomer:catalyst ratio of 1:1 to 10:1 and polymerizing the protDNA to form a pacDNA of predetermined sequence;
6) removing the catalyst;
7) adding a deprotecting agent to remove the protecting groups.

Another aspect of the invention provides a method of synthesizing DNA amphiphiles comprising:
1) reacting one or more hydrophobic, non-polar norbornenyl monomer(s) via ROMP using a monomer:catalyst ratio of 3:1 to 50:1 until the monomers are substantially completely reacted to form a hydrophobic polymer;
2) adding a sub-stoichiometric amount of the protDNA of claim 4;
3) removing the catalyst;
4) adding a deprotecting agent to deprotect the protDNA; and
5) removing unreacted polymers or monomers to thereby isolate the DNA amphiphiles.

Another aspect of the invention provides a protected norbornenyl DNA monomer or protected norbornenyl RNA monomer (protDNA) made by a process comprising the steps of:
1) providing a controlled pore glass (CPG) solid support for oligonucleotide synthesis, wherein the CPG solid support has covalently bound thereon a disulfide linker;
2) conducting a solid-phase oligonucleotide synthesis reaction with ultramild cyanoethyl (CE) phosphoramidites on the CPG solid support to form a protected oligonucleotide having a predetermined nucleotide sequence covalently bound at the 3′ end thereof to the disulfide linker;
3) cleaving the disulfide linker under conditions that release the protected oligonucleotide from the solid support, thereby forming a sulfhydryl group at the 3′end of the protected oligonucleotide; and
4) coupling an iodoacetyl- or a maleimide-functionalized norbornene monomer to the sulfhydryl group of the protected oligonucleotide via a SN₂ or Michael addition reaction, respectively, to produce a norbornenyl protected DNA monomer or norbornenyl protected RNA monomer.

In an embodiment of each of the methods disclosed herein, the heavy metal catalyst is a 3^{rd} generation Grubbs' catalyst. Similarly, in embodiments of each of the methods, the organic solvent is preferably dichloromethane. In other embodiments of each of the methods disclosed herein, methanolic ammonia is a preferred deprotecting agent. In certain embodiments of the methods disclosed herein, the catalyst is removed using EVE (ethyl vinyl ether) after polymerization. In other embodiments of each of the methods disclosed herein using RNA monomers, additional deprotecting agent such as t-butylammonium fluoride in tetrahydrofuran may be used to deprotect silyl protecting groups at the 2′-OH position of the RNA.

### BRIEF DESCRIPTION OF THE DRAWINGS

FIG. 1 is a schematic of the overall scheme of the synthesis and polymerization of the norbornenyl protDNA macromonomer.

FIG. 2a-h show various characterizations of DNA brush homopolymers. (a) PAGE of the raw reaction mixtures after ROMP (monomer:catalyst molar ratio 5:1) and deprotection, showing high conversion. (b) Deprotection conditions and their effect on the retention of brush side chains as indicated by aqueous GPC. (c and d) Aqueous GPC traces and PAGE of DNA bottlebrushes synthesized with increasing monomer:initator feed ratios. (e) Particle morphology of the DNA bottlebrushes observed under TEM. Samples were negatively stained with 2% uranyl acetate. (f) Hydronamic diameter and zeta potential of the DNA bottlebrush. (g) Hybridization kinetics of DNA bottlebrushes with a complementary strand. (h) Thermal melting transitions of duplex DNA bottlebrushes in comparison with free duplex.

FIG. 3 MALDI-TOF MS analysis showing full deprotection of protDNA by treatment with methanolic ammonia.

FIG. 4 is an aqueous GPC chromatogram of DNA monomer (right) and the reaction raw mixture following ROMP of NB-protDNA and deprotection (left; monomer:initiator molar ratio=10:1; deprotected with methanolic ammonia.

FIG. 5a-j. Synthesis and characterization of pacDNAs and DNA deblock amphiphiles. (a) Synthetic sheme of the pacDNA. (b) PAGE of the pacDNAs with 0, 1 and 2 C12 spacers. (c) Typical TEM image of the pacDNA (2× spacer; stained with 2% uranyl acetate), showing a spherical morphology. (d) Hydrodynamic and zeta ppotential of pacDNAs. (e) Enhanced nuclease stability of pacDNAs compared to free DNA. (f) Synthetic sheme of DNA deblock amphiphiles. (g) Spherical micelles assembled from DNA deblock amphiles observed with TEM (2% uranyl acetate staining). (h) Hydrodynamic diameter and zeta potential of the DNA amphiphile micelles. (i) PAGE of the DNA amphiphiles dispersed in PBS. (j) Increased nuclease resistance of the micelles compared with free duplexes.

FIG. 6 is a graph showing the mean fluorescence of cells treated with DNA brushes of varying DP as a function of incubation concentration, as measured by flow cytometry using SKOV-3 cells (total cell counts: 10,000).

FIG. 7 is a graph of relative cellular uptake of pacDNAs as a function of DNA surface densities. Errors (mean±δð) in DNA surface density for polydisperse brushes were calculated based on aqueous GPC results.

FIG. 8 are aqueous GPC chromatograms of pacDNA (DNA-PEG deblock brush) recorded using a PDA detector set at 488 nm (fluorescein component on the DNA). The DNA has 0, 1, or 2 C12 linkers at the 3′ end.

FIG. 9 is a graph showing hybridization kinetics of fluorophore-labeled pacDNAs and free DNA with a complementary quencher strand.

### DETAILED DESCRIPTION

The present disclosure provides a method for the synthesis of high-density DNA and RNA brushes that bypasses the limitations imposed by DNA solubility and nucleobase side reactivities and enables organic-phase manipulation of oligonucleotides. In the methods disclosed herein, the conventional single-step of cleavage/deprotection that conventionally follows solid-phase oligonucleotide synthesis is separated into two separate steps, which allows for the release of nucleobase-protected, charge-masked DNA or RNA strands from the solid support for use in homogenous organic-phase reactions.

Unlike previous methods of generating DNA and RNA brush polymers in organic solvents, which use surfactants to bring oligonucleotides into organics, no surfactant is used in the methods of the present invention. Surfactants are hard to remove and increase the steric bulkiness of an oligonucleotide, making it harder to use longer oligonucleotides for polymerization. Also, unlike previous methods, native phosphodiester oligonucleotides can be used in polymerization. Previous methods generally use peptide nucleic acids for polymerization, to render the oligonucleotides soluble in organics.

The term “oligonucleotides” or “oligos” as used herein includes modified forms as discussed herein as well as those otherwise known in the art. The oligonucleotides used in the various methods disclosed herein may be designed with knowledge of a target sequence or sequences. Methods of making oligonucleotides of a predetermined sequence are well-known. See, for example, Sambrook et al., Molecular Cloning: A Laboratory Manual (2nd ed. 1989) and F. Eckstein (ed.) Oligonucleotides and Analogues, 1st Ed. (Oxford University Press, New York, 1991)) and may be adapted for the methods disclosed herein. Solid-phase synthesis methods are contemplated for both oligoribonucleotides and oligodeoxyribonucleotides (the well-known methods of synthesizing DNA are also useful for synthesizing RNA).

In general, the oligonucleotides synthesized by the present method is from about 3 to about 80 nucleotides in length. It is also contemplated that the oligonucleotide is about 3 to about 75 nucleotides in length, about 3 to about 70 nucleotides in length, about 3 to about 65 nucleotides in length, about 3 to about 60 nucleotides in length, about 3 to about 50 nucleotides in length about 3 to about 45 nucleotides in length, about 3 to about 40 nucleotides in length, about 3 to about 35 nucleotides in length, about 3 to about 30 nucleotides in length, about 3 to about 25 nucleotides in length, about 3 to about 20 nucleotides in length, about 3 to about 15 nucleotides in length, about 3 to about 10 nucleotides in length, and all oligonucleotides intermediate in length of the sizes specifically disclosed to the extent that the oligonucleotide is able to achieve the desired result. Accordingly, oligonucleotides of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 and 80, nucleotides in length are contemplated.

In still other embodiments, oligonucleotides comprise from about 2 to about 25 nucleotides.

The term “protDNA” is used throughout the disclosure to denote protected-DNA oligonucleotide and protected-RNA oligonucleotide unless the context indicates otherwise.

In the present method of solid phase oligonucleotide synthesis, the conventional oligonucleotide deprotection/cleavage step is broken out into two separate steps, enabling the synthesis and isolation of fully protected hydrophobic oligonucleotides. This process enables further manipulation of the protected oligonucleotides in organic solvents. Using this protected form of DNA or RNA, distinct classes of DNA and RNA nanostructures, e.g., DNA/RNA bottlebrushes, pacDNAs, and micellar DNA nanoparticles, can be obtained via standard ring-opening metathesis polymerization (ROMP) in a ‘one-pot’ fashion. These structures represent traditionally difficult-to-synthesize DNA-based structures due to either architectural complexity or amphiphilicity. By turning DNA into an organics-soluble molecule, the disclosed methods can be used to greatly expand the materials space accessible to DNA and enhance existing applications such as DNA-encoded libraries and DNA microarrays.

Disclosed herein are methods of making DNA and RNA oligonucleotides that are soluble in organic solvents, which greatly expands the possibilities for generating DNA and RNA macromonomer-based structures. The disclosed methods provide phosphotriester- and exocyclic amine-protected DNA and RNA that is further modified with a norbornene moiety at the 3′, which enables homopolymerization via ring-opening metathesis to produce bottlebrush-type architectures in high yields. Subsequent deprotection cleanly reveals the unmodified, natural phosphodiester DNA or RNA. The disclosed methods not only achieve high molecular weight DNA and RNA brushes (Mw>380 kDa), but when carried out at low monomer:catalyst ratios, yields oligomers that can be further fractionated to molecularly pure, monodisperse brushes with one to at least ten strands of DNA or RNA or more per brush. The disclosed methods also can be applied to significantly simplify the preparation of traditionally difficult-to-obtain DNA- and RNA-containing structures, such as diblock DNA-polyethylene glycol (PEG) brushes and DNA amphiphiles. With oligos now organics-soluble, a range of DNA and RNA structures is possible, including for example, brush-type poly(oligos), pacOligos (diblock brushes with one block being oligos), linear diblock polymers, multiblock polymers, star, and crosslinked structures.

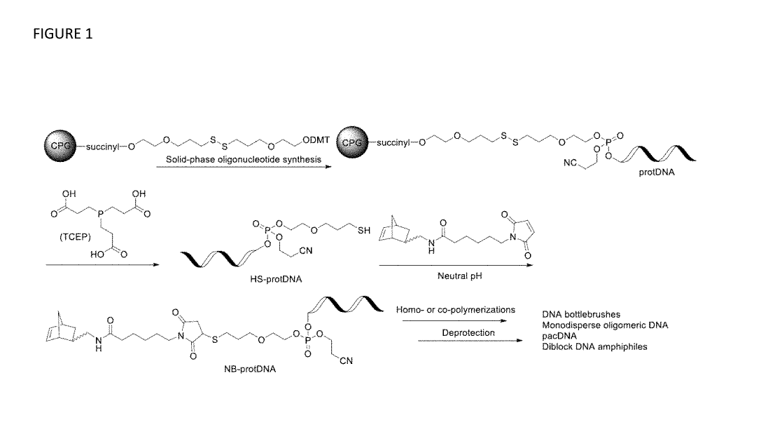

ROMP may be used to convert the norbornene-modified, protected DNA or protected RNA (both referred to herein as “protDNA”) in high yields to distinct classes of polymer-DNA or polymer-RNA nanostructures, such as DNA or RNA bottlebrushes, diblock DNA- or RNA-PEG brushes, and DNA-b-polymer amphiphilic micelles, which represent traditionally challenging DNA and RNA nanostructures for aqueous- or solid-phase techniques (See FIG. 1 for the overall scheme of the synthesis and polymerization of a norbornenyl protDNA macromonomer). The homopolymerization of protDNA not only yields high molecular weight (M_{w}>380 kDa) DNA or RNA brushes but can also be employed to prepare molecularly pure oligomeric DNA or RNA with one to ten strands per molecule when a low monomer:catalyst ratio is used, such as a 1:1 to 3:1 or a 2:1 to 3:1 monomer:catalyst ratio. Previously, oligomeric DNA stars with eight and twelve arms have been painstakingly synthesized via coupling with a careful selection of multivalent cores, followed by several rounds of HPLC purification. (Li, H. et al., Molecular spherical nucleic acids, Proc. Natl. Acad. Sci. USA 115, 4340-4344 (2018)). These monodisperse entities are scientifically valuable structures for accurately interrogating their interactions with living systems. In a single reaction, the protDNA-enabled ROMP disclosed herein fills the gaps of the series of molecular oligomers, including all odd-numbered oligomers.

Synthesis of the norbornenyl protDNA monomer. The ester bond is a common base-labile linkage that is used to tether an oligonucleotide to a controlled pore glass (CPG) solid support during standard oligonucleotide synthesis. Hydrolytic cleavage of the ester also results in the deprotection of the oligonucleotide. In contrast, in the present method, a cleavable linker such as a photocleavabe linker is used in place of the ester bond or a disulfide linker is used in place of the ester, e.g., by using the solid support 3′-Thiol-Modifier 6 S-S CPG (Glen Research) as it can be cleaved under soft reductive conditions, releasing the oligonucleotide in its protected form. For example, Tris-(2-carboxyethyl) phosphine (TCEP) may be used to quantitatively release the protected oligonucleotides from the CPG under weakly acidic conditions, e.g., pH 4.0-6.0. The exposed sulfhydryl group is then used to couple with iodoacetyl- or maleimide-functionalized norbornene monomers, for example, via SN₂ or Michael addition reactions, respectively, to produce the norbornenyl DNA monomers (FIG. 1). Neutral or slightly basic conditions (pH 7.0 to 9.0) (e.g., phosphate buffered saline (PBS), pH=7.4) provides efficient coupling with the iodoacetyl group but may lead to some deprotection of the protDNA (mainly the release of the 2-cyanoethyl group) and loss of solubility in organic solvents. As such, the maleimide-sulfhydryl coupling reaction is preferably used, since it shows no inadvertent deprotection. In certain embodiments of this aspect of the invention, the “ultramild” cyanoethyl (CE) phosphoramidites (phenoxyacetyl dA, 4-isopropyl-phenoxyacetyl dG, dT and acetyl dC, i.e., Pac-dA, iPr-Pac-dG, dT and Ac-dC) are preferable due to a combination of improved solubility and milder deprotection conditions. For RNA synthesis, “ultramild” CE RNA phosphoramidites are preferred. Preferably, at least one spacer, such as a 12-methylene (C12) spacer or a poly(ethylene glycol) linker with 3 to 6 repeating units or other aliphatic linker with from 9 to 18 atoms, is incorporated at the 3′ end to reduce the steric hindrance of the DNA or RNA monomer and facilitate polymerization, although the use of a spacer is not essential. One to ten, preferably one to five and more preferably one or two spacers may be used in each of the embodiments of the invention. Notably, the solubility of protDNA or protRNA in organic solvents is improved by the spacer(s).

The inventors have found that the protected oligonucleotide monomer is compatible with many heavy-metal catalysts such as ruthenium-based catalysts like 2^{nd} or 3^{rd} Generation Grubbs' catalyst, and catalysts such as copper-based and palladium-based catalysts. Unlike natural oligonucleotides, the phosphate backbones and exocyclic amine groups are fully protected in the present oligonucleotide monomers. Since the protected oligonucleotides are organics-soluble, a range of structures is possible with the protected oligonucleotides, including for example, brush-type poly(oligos), pacOligos (diblock brushes with one block being oligos), linear diblock polymers, multiblock polymers, star, and crosslinked structures.

DNA bottlebrush homopolymers. In another aspect of the invention, a process for synthesizing bottlebrush homopolymers is provided. Arranging DNA or RNA into a dense, highly oriented spherical form (spherical nucleic acids, or SNAs) can lead to the emergence of several unusual properties that are absent from linear or cyclic forms of DNA or RNA, such as increased binding affinity to a complementary strand and sharpening of the melting transition (cooperative melting). (Cutler, J. I., Auyeung, E. & Mirkin, C. A. Spherical nucleic acids, J. Am. Chem. Soc. 134, 1376-1391 (2012)). Interestingly, cellular uptake of SNAs is considerably elevated (by 2-3 orders of magnitude) over free DNA or RNA, despite their polyanionic nature. (Cutler et al.) It is not clear, however, whether there exists a distinct threshold density above which multivalent DNA or RNA structures behave like SNAs. The bottlebrush DNA or RNA can in principle achieve SNA-like properties and provide definitive answers to density-related questions as density is a function of the degree of polymerization (DP), which is tunable. While post-polymerization graft-onto chemistries can be used to access a similar architecture, the incomplete grafting due to sterics and the strong charge repulsion ultimately results in limited density. (James, C. R. et al., Poly (oligonucleotide). J. Am. Chem. Soc. 136, 11216-11219 (2014)) The grafting-through synthesis disclosed herein, which uses norbornenyl protDNA can be used to produce the highest possible DNA or RNA density achievable with the brush architecture.

A panel of ROMP conditions was screened for compatibility with protDNA. The results are shown in the tables below.

TABLE 1

| | Monomer | Polymerization |
|---|---|---|
| Solvent | Solubility | Yield |
| N,N-dimethylformamide | High | No reaction |
| Tetrahydrofuran | Low | No reaction |
| Methanol (MeOH) | Low | No reaction |
| Chloroform | Medium | Low |
| Diglyme | Low | No reaction |
| Trifluorotoluene | Low | No reaction |
| Trifluoroethanol | High | No reaction |
| Trifluoro t-butyl alcohol | High | No reaction |
| Dichloromethane (DCM) | Medium | High |
| DCM:MeOH 4:1 v:v | High | No reaction |
| DCM:diglyme 1:1 v:v | Medium | No reaction |

TABLE 2

| | | | “Ultramild” |
|---|---|---|---|
| | Traditional | dA, Ac-dC, | Pac-dA, Ac-dC, |
| Protected Nucleobases | dA, dC, dG, dT | dmf-dG, dT | iPr-Pac-dG, dT |
| Polymerization Yield | Low and harsh | No reaction | High |
| | deprotection | | |
| Grubbs' Catalyst | 1^{st} generation | 2^{nd} generation | 3^{rd} generation |
| Polymerization Yield | No reaction | High | High |
| Effect of C12 Spacers | No Spacer | 1 C12 Spacer | 2 C12 Spacers |
| protDNA solubility in | Low | Medium | High |
| DCM | | | |

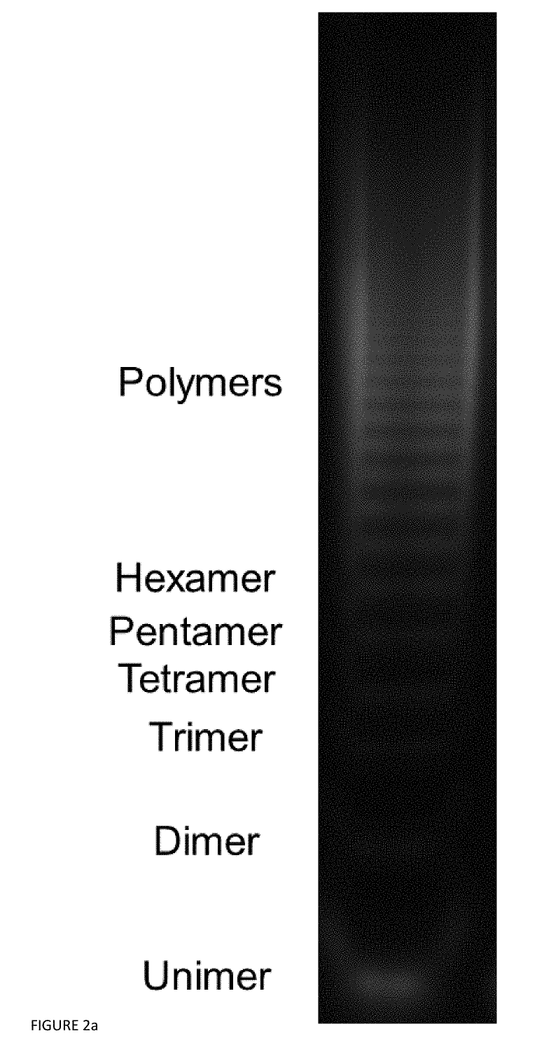

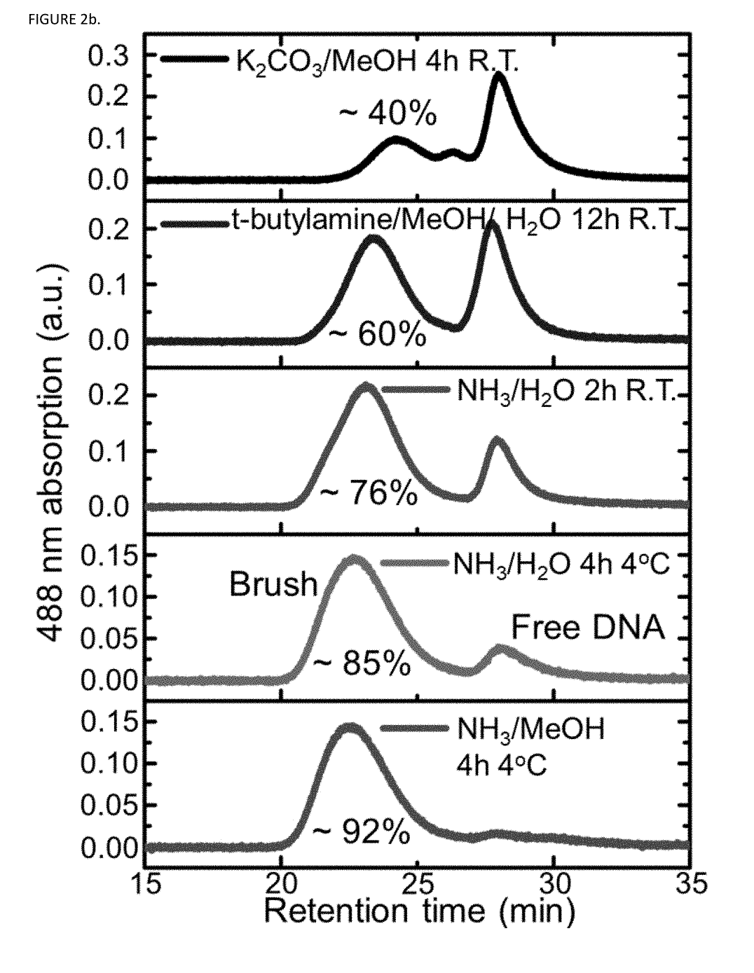

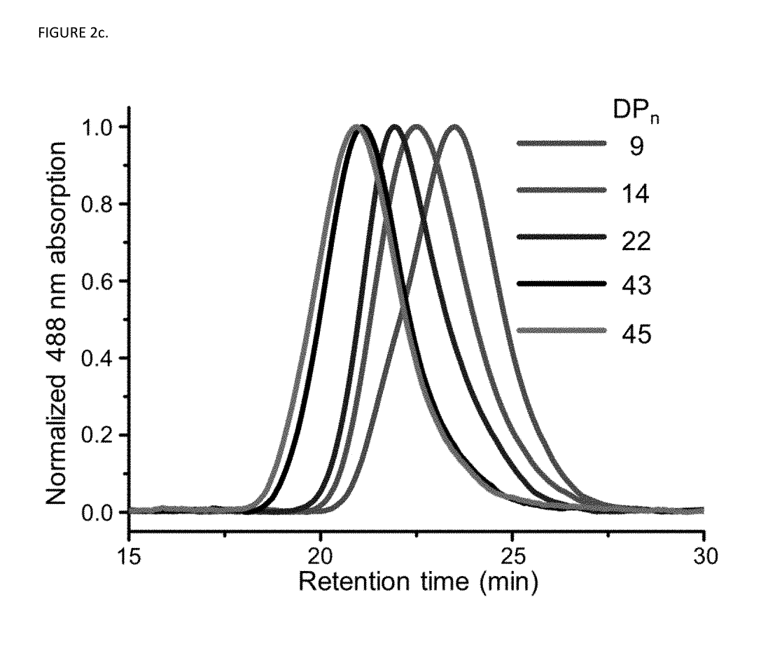

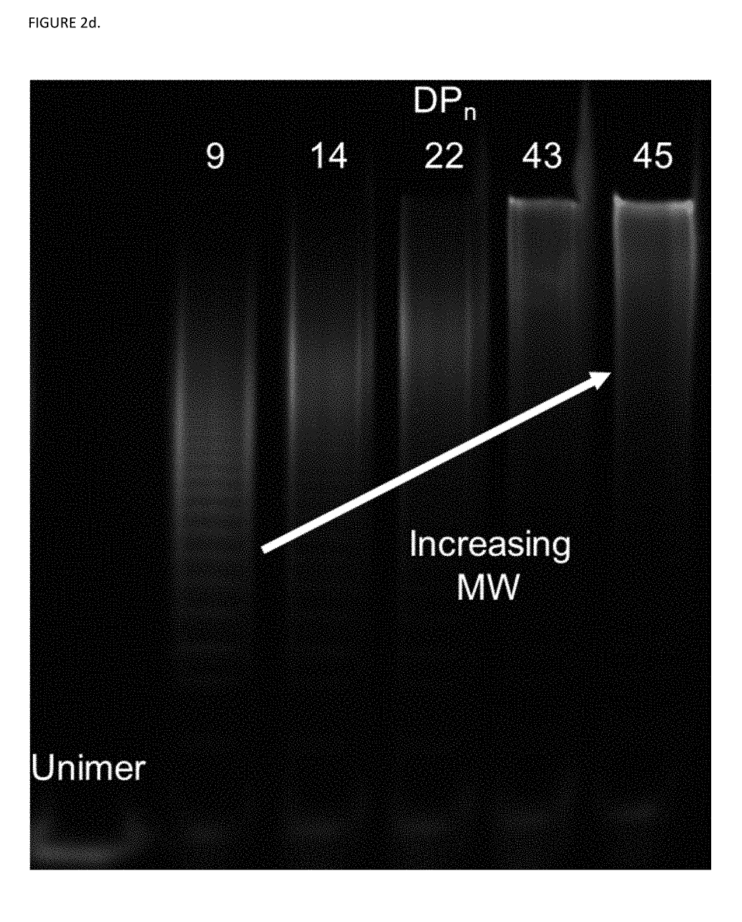

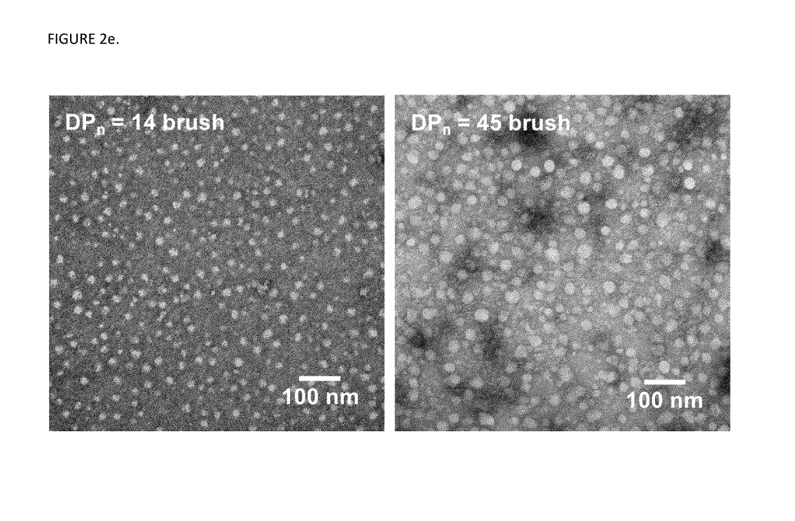

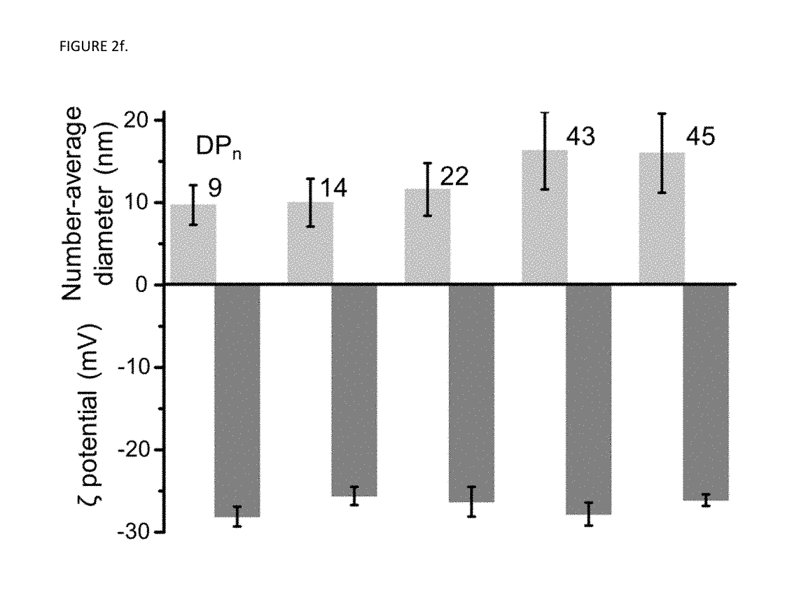

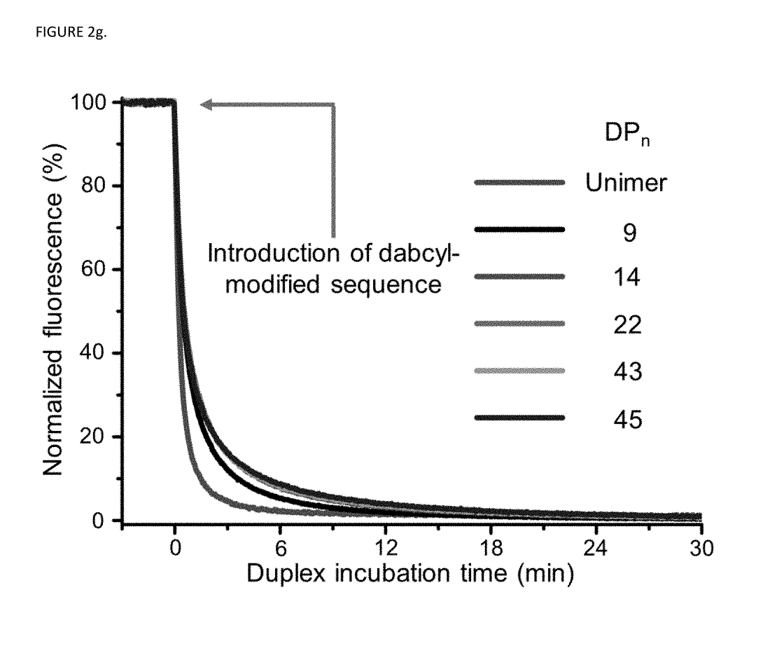

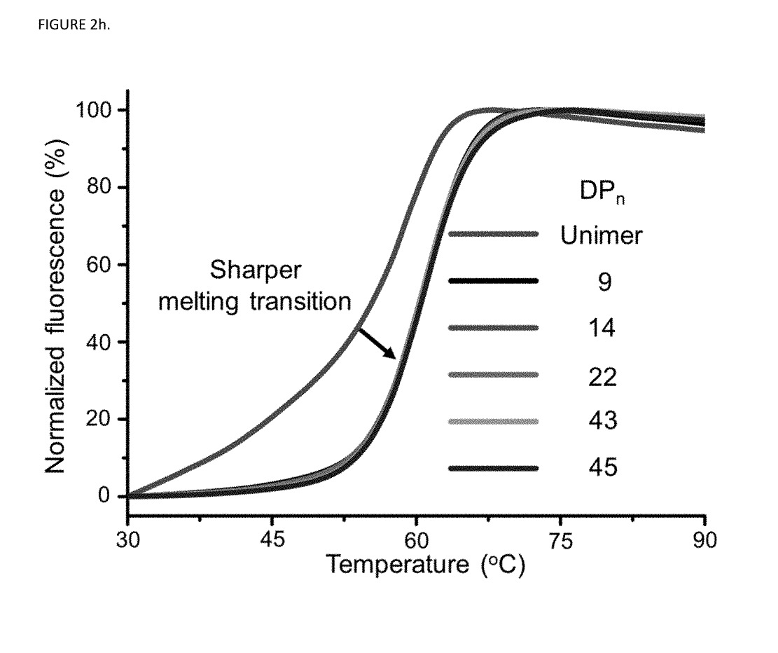

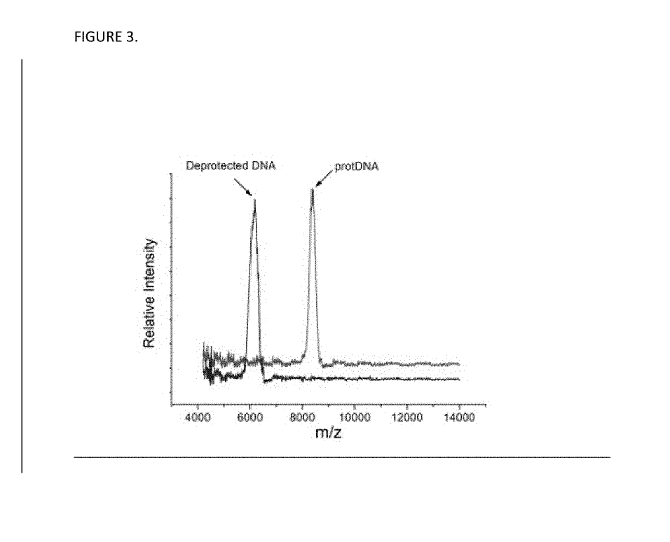

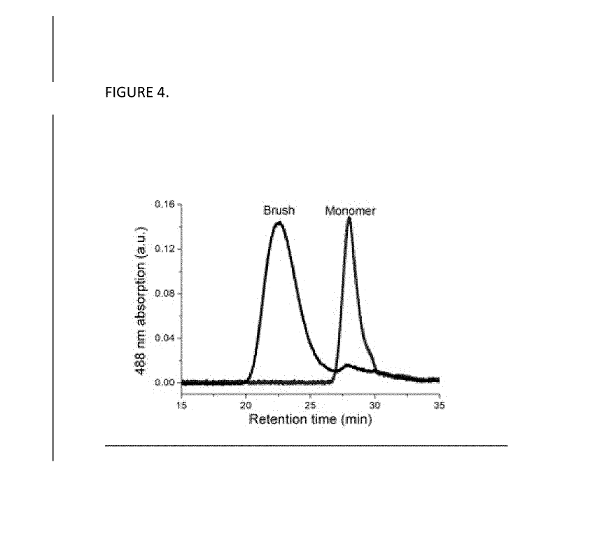

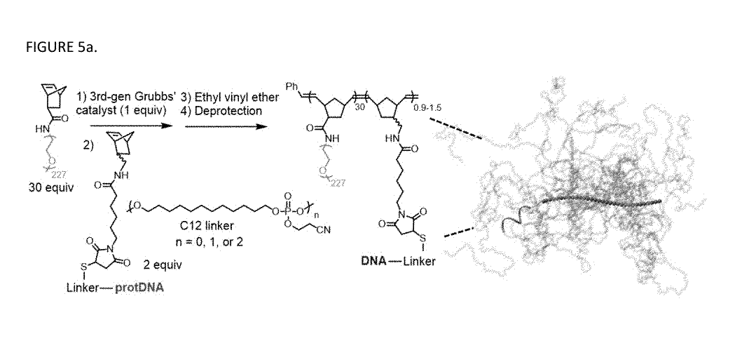

The inventors have found that using 3^{rd}-generation Grubbs' catalyst to initiate the polymerization in dichloromethane (DCM) at −20° C., results in near-quantitative conversion. Thus, a 3^{rd}-generation Grubbs' catalyst is the preferred catalyst for the polymerization step of DNA and RNA bottlebrush homopolymers, although other heavy metal catalysts may be used. Analyzing the raw mixture containing deprotected DNA bottlebrushes by PAGE analysis, a collection of individual bands was observed, which can be assigned to specific degrees of polymerization (FIG. 2a). Various ultramild deprotection conditions (e.g., K₂CO₃ in methanol and t-butylamine/methanol/water) can be used, but some conditions also cleave the brush side chains from the backbone possibly via base-catalyzed retro-Michael reaction (FIG. 2b). Partial cleavage results in lower-density brushes. While cleavage can be achieved using various conditions that are known to those of skill in the art, non-limiting examples of cleavage conditions include 0.05 M potassium carbonate (K₂CO₃) in methanol, 4 h treatment at room temperature; t-butylamine/Methanol/Water=1:1:2 (Vol %), 12 h treatment at room temperature; aqueous ammonia (˜30% wt %), 2 h treatment at room temperature; aqueous ammonia (˜30% wt %), 4 h treatment at 4 degrees Celsius; or methanolic ammonia (˜7 N), 4 h treatment at 4 degrees Celsius, treatment with methanolic ammonia, preferably at 4° C. for 4 h is preferable because it achieves full deprotection with minimal cleavage, as evidenced by GPC and matrix-assisted laser-desorption ionization-time of flight mass spectrometry (FIGS. 2b and 3). The GPC peak for the brush polymer is symmetrical in shape (FIG. 4), indicating that chain-terminating events are much slower compared with propagation, and demonstrates good compatibility of protDNA with 3^{rd}-generation Grubbs' catalyst.

A monomer:catalyst ratio of 2:1 to 250:1, such as 2:1 to 200:1, or 5:1 to 80:1 for example, can be used in the polymerization reaction. Polymerizations with higher monomer:catalyst feed ratios result in a higher degree of polymerization (DP), as detected by GPC and PAGE (FIG. 2c-d), with polydispersity indices (PDIs) in the range of 1.37-1.48. Conversion remains at ˜90% when a 20:1 monomer:catalyst ratio is used, but drops to 68% at 40:1, and 45% at 80:1 (Table 3). Thus, one of skill in the art can select a monomer:catalyst ratio and deprotection conditions to achieve the desired end product.

TABLE 3

| | | | | Number- | |
|---|---|---|---|---|---|
| | | | | Average | |
| | | | Yield | Diameter | ζ Potential |
| Feed Ratio | Mₙ | PD1 | (%) | (nm) | (mV) |
| 5:1 | 8.96 (DPₙ = 9) | 1.48 | 91 | 9.7 ± 2.4 | −28.1 ± 1.2 |
| 10:1 | 14.29 (DPₙ = 14) | 1.45 | 92 | 10.0 ± 2.9 | −25.6 ± 1.1 |
| 20:1 | 22.5 (DPₙ = 22) | 1.40 | 88 | 11.6 ± 3.2 | −26.3 ± 1.8 |
| 40:1 | 42.93 (DPₙ = 43) | 1.40 | 68 | 16.3 ± 4.7 | −27.8 ± 1.4 |
| 80:1 | 45.39 (DPₙ = 45) | 1.38 | 45 | 16.0 ± 4.8 | −26.1 ± 0.7 |
| Free DNA | n.a. | n.a. | n.a. | n.a | −34.5 ± 0.8 |

The reduced conversion at higher monomer:catalyst ratios limited the brush number-average DP to ˜45, which may be attributed to the steric hindrance of the bulky and stiff protDNA macromonomer, and/or the loss of brush solubility as the DP increases. After removing the unreacted unimer DNA from the brush by GPC fractionation, the size and morphology of the brushes were studied by transmission electron microscopy (TEM) and dynamic light scattering (DLS) (FIG. 2e-f). TEM images of negatively stained (with 2% uranyl acetate) brushes showed spherical or elliptical nanoparticles approximately 11-17 nm in diameter. DLS showed increasing number-average hydrodynamic diameters (Dₕ₍ₙ₎ 10-16 nm) with increasing DPₙ, and relatively unchanged ζ potential between −25 and −28 mV, corroborating TEM results.

Monodisperse oligomers and cellular uptake. In another aspect of the invention, a method of making monodisperse oligomers is provided. When carried out at low monomer:catalyst ratios, e.g.,1:1 to 3:1 monomer:catalyst, the ROMP reaction of protDNA is a powerful method to produce molecularly well-defined monodisperse multivalent DNA. Octameric and dodecameric DNA molecules have been meticulously synthesized previously by coupling linear DNA strands to a well-defined multivalent core, followed by several rounds of chromatographic purifications to remove partially coupled products. The monodisperse structures are poised to enable accurate interrogation of their interactions with living systems. However, due to the need to use a variety of cores to access a full numeric series of oligomers and the difficulty in synthesizing odd-valency cores, there has not been a conclusive study correlating cellular uptake and DNA density. The ability to synthesize molecularly well-defined monodisperse multivalent DNA via the disclosed method overcomes the obstacles of the prior methods.

DNA-Polymer diblock brush polymers. In another aspect of the invention there is provided a method of synthesizing DNA-polymer brushes and RNA-polymer brushes, such as DNA-polymer diblock brushes. As used herein, the term “brush polymer” means a polymer having an array of macromolecular polymer chains attached to the polymer backbone in sufficient proximity so that the unperturbed solution dimensions (in a good solvent) of the chains are altered. Furthermore, this close proximity causes overlap of adjacent chains and thus significantly alters the conformational dimensions of individual polymer chains such that they extend or alter their normal radius of gyration to avoid unfavorable interactions.

Any biocompatible non-cationic polymer that does not interact with protein (e.g., exhibits stealth properties which enable it to avoid recognition by liver receptors and other proteins) can be used to generate the brush polymer component of the DNA-polymer or RNA-polymer. (See Laschewsky, A., Polymers, 2014, 6, 1544-1601, incorporated herein). For example, polyethylene glycol (PEG) polymers may be used, as well as polysaccharides such as amylose and zwitterion polymers such as poly(methacryloyl-L-lysine), poly(sulfobetaine methacrylate) and poly(carboxybetaine methacrylate).

The brush polymer component of the DNA or RNA brush polymers may be a homopolymer, di-block copolymer, a tri-block copolymer, etc., e.g., where one or more blocks are attached with oligonucleotides and the other block(s) form the sidechains. All of the blocks together form the backbone of the brush polymer. The brush polymer consists of a backbone and side chains. Many side chains are tethered to the backbone in close proximity. The backbone can be a homopolymer or a copolymer (diblock, multiblock, or a random mixture of different monomers). The side chain can also be a homopolymer such as PEG, or a copolymer. The side chain may have a different composition from the backbone. For example, the backbone may be polynorbornene and the side chains may be homopolymeric units of PEG, or a zwitterionic polymer or a polysaccharide. In all cases the polymer is non-cationic and biocompatible. Preferably, the polymer component of the DNA-brush polymer is a PEG polymer or another polymer having PEG-like properties, e.g., poly(carboxybetaine methacrylate).

Covalent attachment of poly(ethylene glycol) (PEG) remains a widely used technique to impart better pharmacokinetic properties to many forms of therapeutics. However, a single linear or slightly branched PEG, even with high molecular weight (40-100 kDa), cannot sufficiently shield oligonucleotides from interaction with serum or cell membrane proteins to provide appropriate biopharmaceutical properties for systemic use. (Lu, X. & Zhang, K., PEGylation of therapeutic oligonucletides: From linear to highly branched PEG architectures, Nano Research 11, 5519-5534, (2018)). Recently, the inventors developed a new form of PEGylated oligonucleotide, termed pacDNA (polymer-assisted compaction of DNA), which consists of oligonucleotides tethered to the backbone of bottlebrush-architectured PEG or other similar polymer having PEG-like properties, e.g., poly(carboxybetaine methacrylate), with many (typically >25) shorter (5-10 kDa) PEG (polymer) chains. (US 2018/0230467, incorporated herein in its entirety) The highly branched PEG architecture provides a “Goldilocks” PEG density: high enough to prevent protein access but not too high to impair DNA hybridization with a complementary sequence. The selectivity of the pacDNA in choosing a binding partner greatly reduces unwanted side effects and enhances pharmacokinetics. The current synthesis of pacDNA involves sequential copolymerization and post-polymerization modifications to obtain a diblock brush polymer, poly(oxanorbornenyl-azide)-b-poly(norbornenyl PEG), onto which dibenzocyclooctyne (DBCO)-functionalized strands are coupled via copper-free click chemistry.

Using the present method which enables the manipulation of protDNA in organic solvents, it is now possible to prepare pacDNA or pacRNA in one-pot by sequential copolymerization of norbornenyl PEG (or other norbornenyl polymer with properties similar to PEG) and protDNA or protRNA. For example, norbornenyl PEG (10 kDa) or other polymer with PEG-like properties is polymerized as the first block using a heavy metal catalyst such as a 3r^{d}-generation Grubbs' catalyst in a ROMP reaction (Bielawski, C. W. & Grubbs, R. H. Living ring-opening metathesis polymerization. Prog. Polym. Sci. 32, 1-29 (2007), incorporated herein in its entirety) with a monomer:catalyst molar ratio of 10:1 to 100:1 (e.g., 30:1). The selection of catalyst and ratio of monomer to catalyst can be adjusted as discussed above. Upon completion of the polymerization of the first block (which can be monitored by GPC, for example), protDNA monomers are introduced to the ROMP reaction mixture at a low monomer:catalyst ratio, such as 1:1 to 10:1 (monomer:catalyst) or 2:1 to 5:1 for example. Preferably, at least one spacer and more preferably two spacers, such as a C12 methylene spacer is attached to the 3′ end of each protDNA monomer.

Diblock amphiphiles with DNA as the hydrophilic segment. In another aspect of the invention there is provided a method of synthesizing DNA amphiphiles. DNA amphiphiles are another class of difficult-to-access structures using homogeneous coupling, especially those with a non-polar hydrophobic segment. Synthesis of diblock amphiphiles using the protDNA of the invention is carried out as follows:

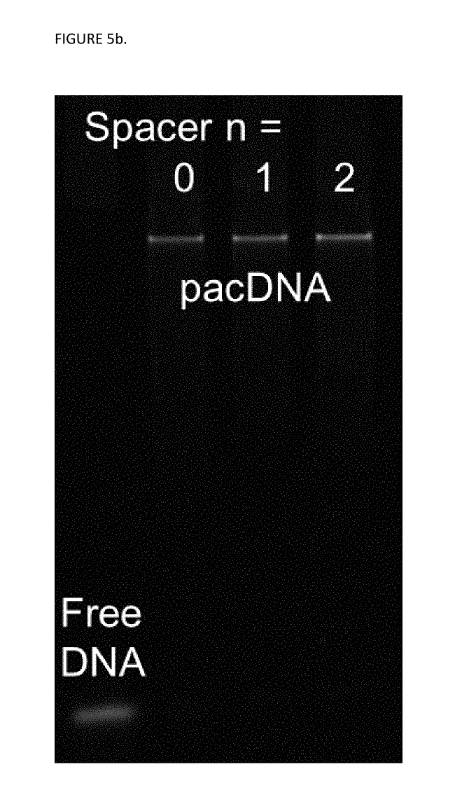

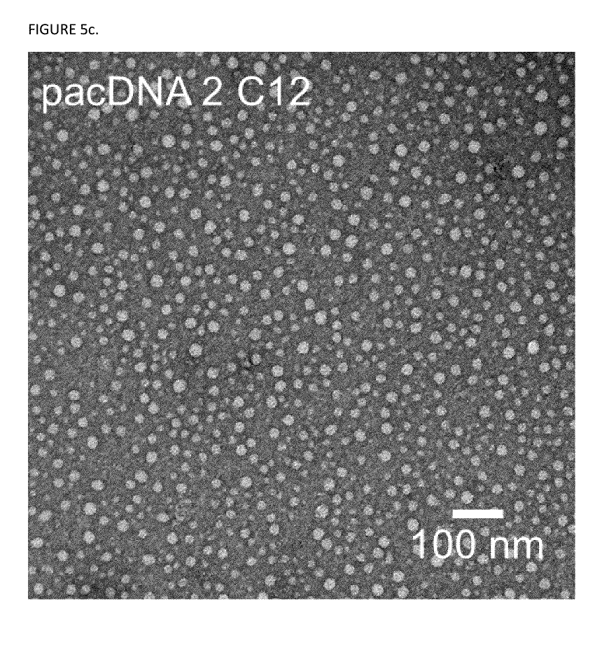

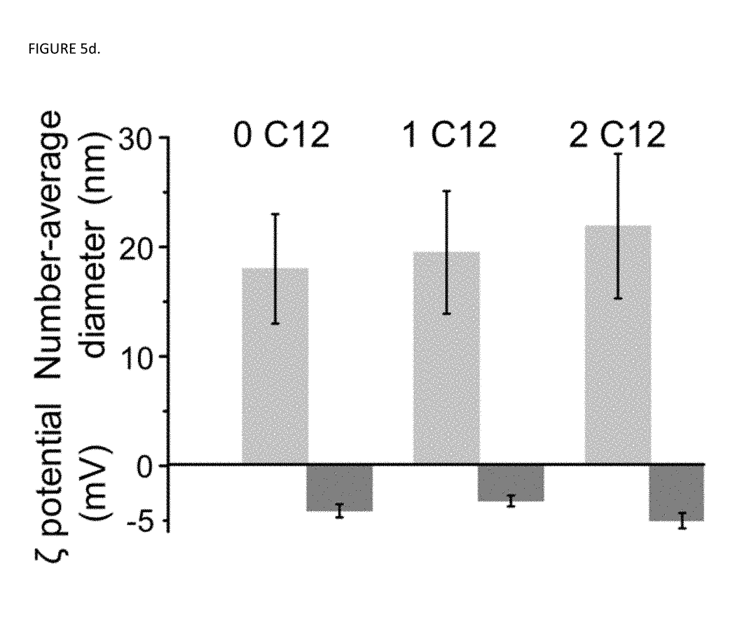

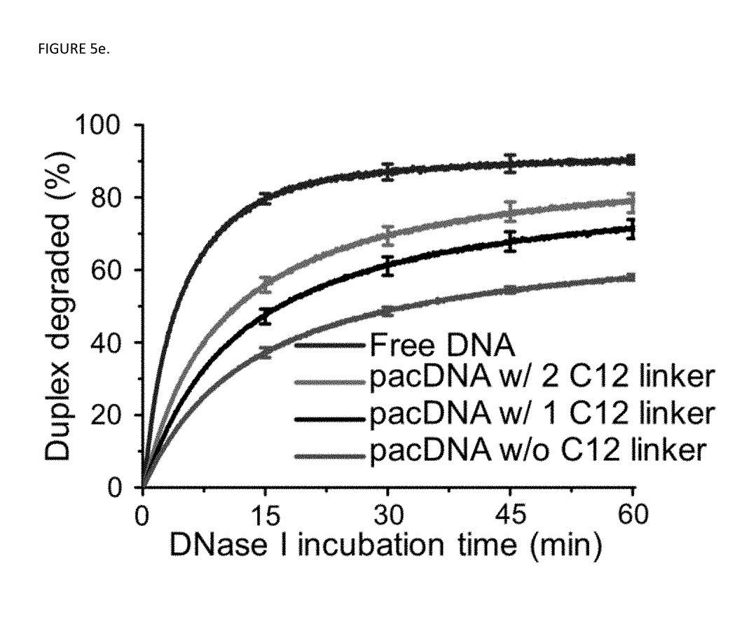

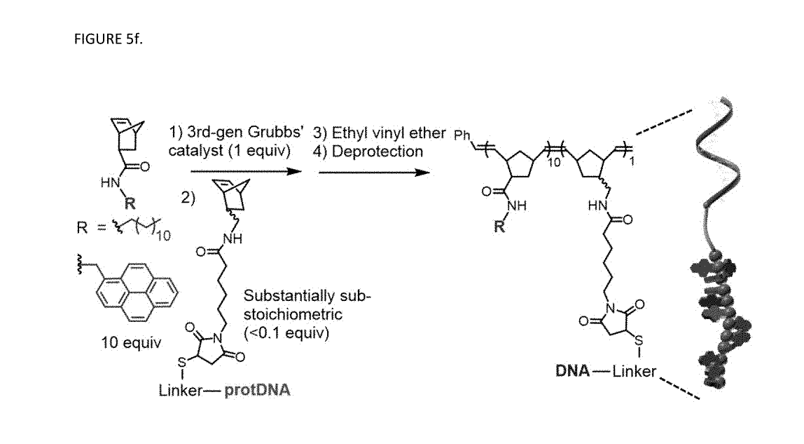

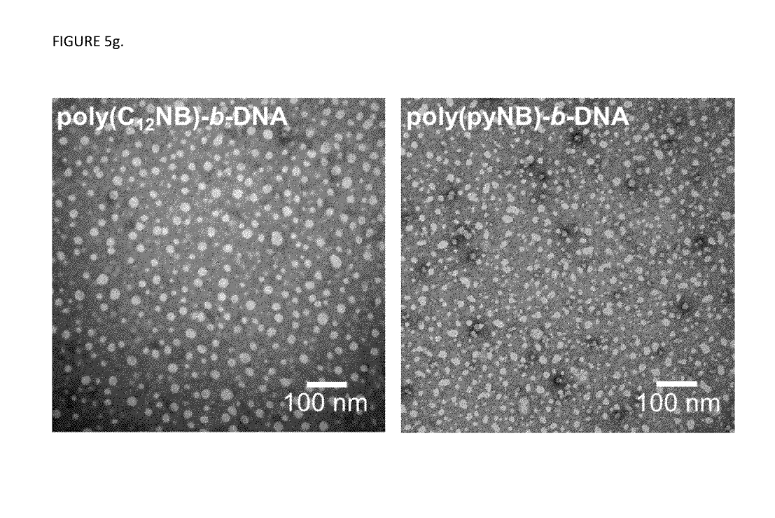

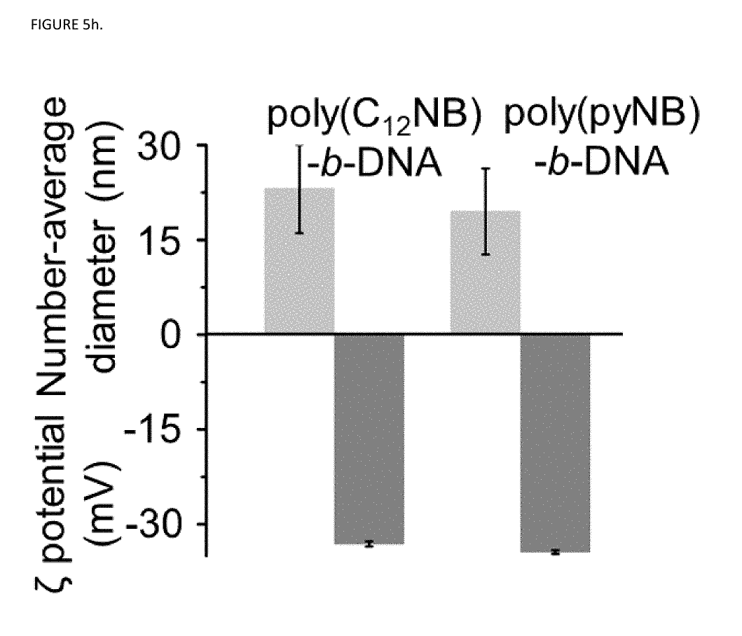

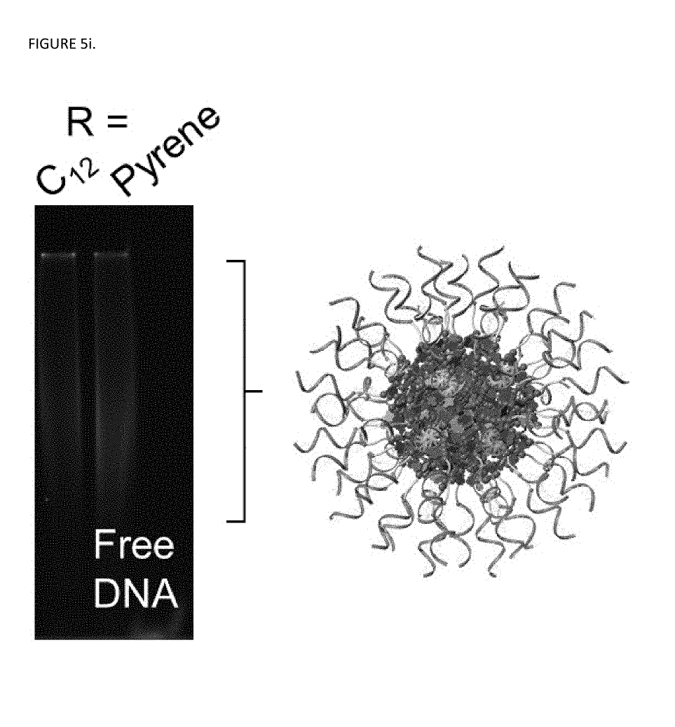

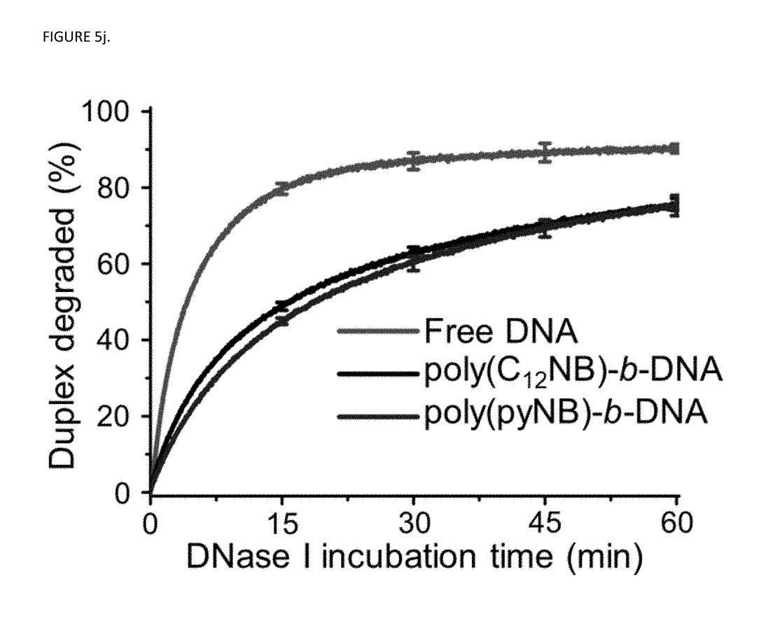

Two hydrophobic, non-polar norbornene-functionalized monomers, such as norbornenes carrying an aliphatic chain, e.g., C₁₂NB, and pyrene, pyNB are reacted via ROMP to form a hydrophobic segment (See FIG. 5f). A monomer:catalyst ratio is selected to balance the amphiphilicity of the final amphiphile so that the micelles are formed in an aqueous buffer, e.g. a monomer:catalyst ratio of 10:1. Once the monomers are consumed (this can be monitored by TLC, for example), a substantially sub-stoichiometric amount of protDNA monomers is added to the mixture, e.g., protDNA monomer:catalyst of 1:10. The large excess of the growing polymer chains ensures that amphiphiles containing only one DNA strand are statistically most probable to form. After removal of the chain-end catalyst with a chain-end capping agent such as ethyl vinyl ether (EVE), for example and deprotection of the protDNA with methanolic ammonia, for example, unreacted polymers are removed by precipitation in water. Non-micellar residues can be removed by aqueous GPC.

In each of the synthesis methods described herein, the synthesis products may be detectably labelled. Detectable labels include fluorescein by using 5′-fluorescein phosphoramidite (Glen Research), for example.

Kits. Another aspect of the invention provides for kits including a plurality of protected norbornenyl DNA monomers (protDNA) including -dA, -dG, -dC and -dT. The kits may further contain reagents for synthesis of oligonucleotides. In one embodiment of this aspect, the kit contains at least one type of DNA-brush polymer as described herein or a plurality of types of DNA-brush polymers providing a plurality of different oligonucleotides as described herein attached to a brush polymer backbone. In some embodiments of the kits provided, oligonucleotides include a detectable label or the kit includes a detectable label which can be attached to the oligonucleotides or the brush polymer.

### EXAMPLES

Materials and Methods

Phosphoramidites and supplies for DNA synthesis were purchased from Glen Research Co. ω-amine terminated poly(ethylene glycol) methyl ether (Mₙ=10 kDa, PDI≈1.1) was purchased from JenKem Technology. Dulbecco's Modified Eagle Medium (DMEM) was purchased from Sigma-Aldrich Co. Human SKOV-3 cancer cell line was purchased from American Type Culture Collection (Rockville, Md., USA). All the other common materials were purchased from Sigma-Aldrich Co., VWR International LLC., or Fisher Scientific Inc., and used without further purification unless otherwise indicated. Dynamic Light Scattering (DLS) and ζ potential data were acquired on a Zetasizer Nano-ZSP (Malvern Panalytical, Ltd, UK). Fluorescence spectroscopy was performed on a Cary Eclipse fluorescence spectrophotometer (Varian Inc., CA, USA). ¹H and ¹³C NMR spectra were recorded on Varian 400 MHz or Varian 500 MHz spectrometers (Varian Inc., CA, USA). Chemical shifts (δ) were reported in ppm. GPC measurements for aqueous samples were performed on a Waters Breeze 2 GPC system equipped with an Ultrahydrogel™ 500 column and two Ultrahydrogel™ 250, 7.8×300 mm columns connected in series as well as a 2998 PDA detector (Waters Co., MA, USA). Sodium nitrate aqueous solution (0.1 M) was used as the eluent running at a flow rate of 0.8 mL/min. The number- and weight-average molecular weights of a polymer sample were calculated based upon sodium polystyrene sulfonate (PSSNa) standards, while polydispersity indices (PDIs) were determined using monodisperse DNA oligomers as standards, assuming each oligomer has a PDI of 1.01. Reverse phase HPLC was performed using a Waters Breeze 2 HPLC system coupled to a Symmetry 3.5 μm, 4.6×75 mm C18 column and a 2998 PDA detector (Waters Co., MA USA), using Nanopure™ water and HPLC-grade acetonitrile as mobile phases. Gel electrophoresis was performed using 4-20% Mini-PROTEAN® TGX Stain-Free™ Protein Gels (Bio-Rad Laboratories, Inc., CA, USA) in 0.5× tris/borate/EDTA (TBE) buffer with a gradient voltage from 70V to 180V. Gel images were acquired on an Alpha Innotech Fluorchem Q imager. TEM samples were imaged on a JEOL JEM 1010 electron microscope utilizing an accelerating voltage of 80 kV. Matrix-assisted laser desorption-ionization time of flight mass spectrometry (MALDI-TOF MS) measurements were carried out on a Bruker Microflex LT mass spectrometer (Bruker Daltonics Inc., MA, USA). Electrospray ionization (ESI) MS measurements was performed with the following parameters. Solvent: acetonitrile/water with 0.1% formic acid additive, 2 ml/min flow rate; column: SunFire C18 column, 100 Å, 3.5 μm, 4.6 mm×50 mm with a SunFire C18 Sentry Guard Cartridge, 100 Å, 3.5 μm, 4.6 mm×20 mm; detector: Waters 2996 PDA detector; HPLC: Waters 2795 separations module; mass spectrometer: Waters Micromass ZQ.

Synthesis of the protDNA macromonomer. Solid-phase oligonucleotide synthesis was conducted following standard protocols. After the completion of the synthesis (1 μmol scale), the CPG was air-dried and suspended in a solution containing 800 μL of acetonitrile and 400 μL of aqueous cleavage buffer (25 mg of TCEP.HCl and 15 mg of NaHCO₃ in 1960 μL Nanopure water™). After shaking overnight at room temperature, 400 μL of Nanopure water™ was added to the solution and the CPG solid support was removed by filtration through a 0.45 μm polytetrafluoroethylene (PTFE) filter. The filtrate was then subjected to reverse-phase HPLC to separate thiolated protDNA monomer (C18 column; mobile phases: acetonitrile and pure water; flow rate: 1.0 mL/min; gradient: constant 50/50 vol % of acetonitrile/water from 0 to 5 min, then 100% acetonitrile for 30 min). The product was lyophilized and redissolved in a solution consisting of 500 μL of acetonitrile and 500 μL of water. Next, 6 mg of the norbornenyl maleimide was added to the solution, and the mixture was vigorously shaken at 4° C. for 12 h. Last, the mixture was filtered and subjected to reverse-phase HPLC to separate the norbornenyl protDNA monomer from residues using the same gradient. Notably, the product exhibited multiple peaks in HPLC, which may be attributed to the chiral phosphate centers in the DNA backbone. The final solution was lyophilized to yield the protDNA monomer as a white powder.

Estimation of the DNA surface density. Density is measured at the outer periphery of the nanoparticle as opposed the footprint where DNA meets with the polymer backbone. To establish a model to estimate the DNA surface density for brushes of any DP, it is assumed that all chemical bonds in the polymer backbone and linkers are in the fully stretched conformation and occupy the maximum three-dimensional space. With this assumption, the central polymer backbone can be treated as a cylinder with the radius of R+r and a length of (n−1)×L, where R is the length of the DNA, r is the length of the linker, and L is the length of the repeating unit in the backbone. The DNA surface density is therefore a function of DP:

$$DNA surface density ( pmol \text{/} {cm}^{2} ) = \frac{n}{2 π ( R + r ) ( n - 1 ) L + 4 π ( R + r ) 2}$$

where r and L are estimated to be 5.79 nm and 0.60 nm, respectively. The DNA has a 15-bp sequence and thus R=0.34 nm×15=5.1 nm.

dsDNA Melting Transitions

Fluorescein-labeled ssDNA (10 μL, 20 μM) samples were mixed with complementary dabcyl-labeled DNA (10 μL, 20 μM) in 980 μL of PBS solution to pre-form dsDNA. To anneal the duplex, the mixtures were heated to 90° C. and cooled slowly to room temperature overnight in a thermally sealed container. The melting curves of the duplexes were recorded on a quantitative polymerase chain reaction (qPCR) instrument (CFX96 Touch™ Real-Time PCR Detection System, Biorad, USA). The temperature range was 30° C. to 90° C. and data were collected in 0.2° C. increments. All measurements were performed in triplicates.

DNA Nuclease Stability

dsDNA was pre-formed by mixing fluorescein-labeled DNA (1.0 eq., 50 nM) with dabcyl-labeled complementary strand (2.0 eq.) in DNase I buffer (10 mM tris, 2.5 mM MgCl₂, and 0.5 mM CaCl₂, pH=7.5). After gently shaking overnight, 800 μL of the dsDNA solution was transferred to a quartz cuvette. The starting fluorescence was recorded for 3 min (excitation: 490 nm, emission: 520 nm), before the addition of DNase I (0.08 unit) and rapid and thorough mixing. The fluorescence was monitored every 3 sec for 60 min. To determine the endpoints, an additional 5 units of DNase I was added to each sample and 12 h was given to ensure complete degradation of the dsDNA. All the results were normalized by treating starting points as 0% degradation and endpoints as 100% degradation. The measurements were performed in triplicates.

Confocal Laser Scanning Microscopy

SKOV-3 cancer cells were seeded at a density of 1.0×10⁵ cells/well in 24-well glass bottom plates and were cultured overnight in full growth DMEM supplemented with 10% fetal bovine serum (FBS), 1× GlutaMAX, and 1× Antibiotic-Antimycotic at 37° C. in 5% CO₂ atmosphere. After removing the medium, the cells were rinsed with PBS buffer twice and serum-free medium containing fluorescein-labeled free DNA or DNA brushes with an equal dose of total DNA concentration (800 nM) were added to each well, followed by further incubation at 37° C. for 4 h. The cells were then rinsed with PBS buffer twice, fixed with 4% formaldehyde solution for 20 min, stained with 10 μM DAPI for 2 min, and rinsed with PBS twice. The cells were imaged by confocal laser scanning microscopy (Leica, UK) under identical imaging settings at excitation wavelengths of 408 nm (DAPI), and 488 nm (fluorescein).

Flow Cytometry

SKOV-3 cancer cells were seeded with a density of 3.0×10⁵ cells/well in 12-well plates and were cultured overnight in full growth DMEM medium supplemented with 10% FBS, 1× GlutaMAX, and 1× Antibiotic-Antimycotic at 37° C. in 5% CO₂ atmosphere. After removing the medium, the cells were rinsed with PBS buffer twice and serum-free medium containing fluorescein-labeled free DNA or DNA brushes with varying doses of total DNA concentration (0-800 nM) were added to each well, followed by further incubation at 37° C. for 4 h. After removing the medium, the cells were rinsed with PBS buffer twice, trypsinized, centrifuged, re-suspended in fresh PBS buffer, and analyzed by flow cytometry (FACS Calibur, BD Bioscience, San Jose, Calif., USA) to determine the extent of cell uptake (cell counts: 10,000).

Unless otherwise indicated, the following DNA macromonomer was used in the polymerization studies disclosed in the Examples: 5′-fluorescein-CTC CAT GGT GCT CAC-(C12)₂-norbornene-3′ (SEQ ID NO.:1). Electrospray ionization-mass spectrometry (ESI-MS) confirmed the successful synthesis of the DNA monomer (exact mass: 8252.37 Da, calc. 8260.35 Da).

### Example 1

### Synthesis of Norbornene-Maleimide Linker, 1

DCC

6-Maleimidohexanoic acid (0.50 g, 2.37 mmol) and N,N′-dicyclohexylcarbodiimide (DCC, 0.59 g, 2.84 mmol) were dissolved in 5 mL of dichloromethane (DCM). In a separate vial, 5-norbornene-2-methylamine (mixture of isomers) (0.29 g, 2.37 mmol) was dissolved in 1 mL of DCM, and was added dropwise to the first vial containing the mixture. The reaction mixture was allowed to stir for 1 h at room temperature, followed by filtration to remove the urea byproduct. The filtrate was then concentrated and purified by silica gel column chromatography (hexane: ethyl acetate=2:1, v/v). The solvent was subsequently removed under reduced pressure to yield 1 as white solid (0.58 g, 77%). ¹H-NMR (400 MHz, CDCl₃): δ 6.65 (s, 2H), 6.11-6.14 (dd, J=2.7 Hz, 1H), 5.90-5.92 (dd, J=2.7 Hz, 1H), 5.79 (s, 1H), 3.45-3.50 (t, J=7.0 Hz, 2H), 2.83-3.01 (m, 2H), 2.76 (s, 2H), 2.11-2.16 (t, J=6.9 Hz, 2H), 1.76-1.83 (m, 1H), 1.53-1.67 (m, 6H), 1.38-1.41 (m, 1H), 1.25-1.32 (m, 2H), 1.19-1.22 (m, 1H); ¹³C-NMR (400 MHz, CDCl₃): δ 172.56, 170.65, 137.33, 133.96, 131.99, 49.28, 44.01, 43.21, 42.20, 38.63, 37.44, 36.19, 29.94, 28.13, 26.21, 25.15.

### One-Pot Example 2

### Synthesis of Norbornene-C12 Monomer, 2

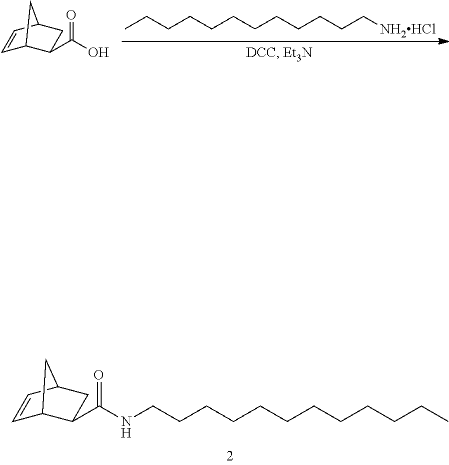

Exo-5-norbornene carboxylic acid (0.50 g, 3.62 mmol) and DCC (0.90 g, 4.36 mmol) were dissolved in 4 mL of DCM. In a separate vial, dodecylamine (812 μL, 0.67 g, 3.61 mmol) was dissolved in 2 mL of DCM, which was added dropwise to the first vial containing the mixture. The reaction mixture was allowed to stir for 1 h at room temperature, followed by filtration to remove the urea byproduct. The filtrate was then concentrated and purified by silica gel column chromatography (hexane:ethyl acetate=99:1 to 95:1, v/v). The solvent was removed under reduced pressure to yield 2 as white solid (0.81 g, 73%). ¹H-NMR (400 MHz, CDCl₃): δ 6.10-6.13 (dd, J=2.8 Hz, 1H), 6.07-0.09 (dd, J=2.8 Hz, 1H), 5.79 (s, 1H), 3.21-3.75 (t, J=6.6 Hz, 2H), 2.89-2.92 (m, 2H), 1.98-2.02 (m, 1H), 1.87-1.91 (dt, J=7.8, 3.4 Hz, 1H), 1.69-1.71 (d, J=8.3 Hz, 1H), 1.46-1.50 (m, 2H), 1.21-1.35 (m, 20H), 0.85-0.88 (t, J=7.0 Hz, 3H); ¹³C-NMR (400 MHz, CDCl₃): δ 174.90, 138.18, 136.01, 47.18, 46.33, 44.66, 41.55, 39.73, 31.89, 30.51, 29.69, 29.62, 29.61, 29.56, 29.52, 29.32, 29.29, 26.92, 22.66, 14.10.

### Example 3

### Synthesis of Norbornene-Pyrene Monomer, 3

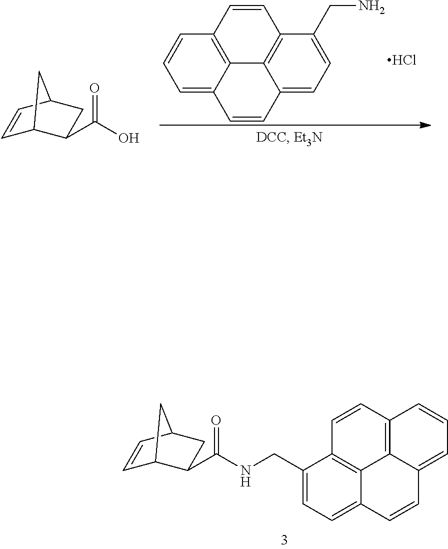 [SMILES: O=C(O)C1CC2C=CC1C2 ; NCc1ccc2ccc3cccc4ccc1c2c34 ; Cl ; O=C(NCc1ccc2ccc3cccc4ccc1c2c34)C1CC2C=CC1C2]

Exo-5-norbornene carboxylic acid (0.50 g, 3.62 mmol) and DCC (0.90 g, 4.36 mmol) were dissolved in 4 mL of DCM. In a separate vial, 1-pyrenemethylamine hydrochloride (0.97 g, 3.62 mmol) and triethylamine (520 μL, 0.38 g, 3.73 mmol) were dissolved in 2 mL of DCM, and the solution was added dropwise to the first vial containing the mixture. The solution was stirred at room temperature for 2 h. After filtration to remove the urea byproduct, the filtrate was washed with saturated sodium bicarbonate solution, 1 M HCl solution, brine, and then dried over sodium sulfate. The crude product was concentrated and purified by silica gel column chromatography (hexane: ethyl acetate=4:1, v/v). The solvent was subsequently removed under reduced pressure to yield 3 as slightly yellow solid (0.53 g, 42%). ¹H-NMR (400 MHz, CDCl₃): δ 8.10-8.24 (m, 5H), 7.99-8.07 (m, 3H), 7.92-7.95 (d, J=7.6 Hz, 1H), 6.09-6.12 (dd, J=2.8 Hz, 1H), 6.00-6.03 (dd, J=2.8 Hz, 1H), 5.89 (s, 1H), 5.06-5.19 (m, 2H), 2.92-2.96 (d, J=9.7 Hz, 2H), 1.97-2.02 (m, 2H), 1.80-1.83 (m, 1H), 1.37-1.40 (m, 1H), 1.29-1.33 (m, 1H); ¹³C-NMR (400 MHz, CDCl₃): δ 175.18, 138.25, 135.92, 131.38, 131.23, 131.22, 130.74, 129.04, 128.19, 127.54, 127.32, 127.25, 126.10, 125.39, 125.34, 125.03, 124.74, 124.69, 122.91, 47.25, 46.42, 44.78, 42.19, 41.61, 30.55.

### Example 4

### Concentration Determination for the protDNA Macromonomer

An aliquot of HPLC-purified protDNA macromonomer was dried under vacuum, followed by deprotection using methanolic ammonia for 4 h at 4° C. After the complete removal of methanol and ammonia, 1 mL of 1× phosphate buffered saline (PBS) buffer was added to dissolve the deprotected DNA monomer. The amount of protDNA in the aliquot was calculated based on the fluorescence of the deprotected DNA using a calibration curve established with fluorescein isothiocyanate (FITC) standard.

### Example 5

### General Method for the Synthesis of DNA Bottlebrush Homopolymers

DNA bottlebrush homopolymers were synthesized via ring-opening metathesis polymerization (ROMP) of protDNA using 3^{rd}-generation Grubbs' catalyst. The catalyst and protDNA monomer (100-300 nmol) were dissolved in anhydrous DCM in two separate vials under inert atmosphere. The vial containing the protDNA monomer (1.0 eq.) was then cooled to −20° C. using an ice-salt water bath, to which a solution of Grubbs's catalyst (0.0125-0.2 eq.) was added via a microsyringe. The reaction mixture (˜500 μL DCM) was maintained at −20° C. for 5 min, transferred to an ice bath (0° C.), and slowly warmed to room temperature overnight. To terminate the polymerization, 150 μL of ethyl vinyl ether (EVE) was added to the vial and the reaction mixture was stirred for another 2 h. After removing the solvent under reduced pressure, the product was deprotected by treatment with 1 mL of methanolic ammonia (ca. 7 N) solution at 4° C. for 4 h. The ammonia/methanol was then removed under reduced pressure. The solid was re-dissolved in 1 mL of water and subjected to aqueous GPC. Fractions containing the brush product was collected and dialyzed against Nanopure™ water to remove residues. The final solution was lyophilized to yield DNA bottlebrush homopolymers as a yellow powder.

### Example 6

### Synthesis and Purification of Monodisperse DNA Oligomers

To produce oligomers as the primary product, the polymerization of protDNA monomers was carried out with the monomer:catalyst ratio of 2:1. After polymerization and deprotection, the DNA brushes were redissolved in 10% glycerol aqueous solution. Oligomers with different degrees of polymerization were separated by polyacrylamide gel electrophoresis (running buffer: 0.5× TBE buffer; Bio-Rad precast Any-kD™ polyacrylamide gel, prep-well comb, 7 cm strip, 450 μL). After oligomers exhibited efficient separation, the gel was cut by a razor into strips containing different oligomers. The gel strips were further cut into smaller pieces and soaked into 0.5× TBE buffer. After gentle shaking for 1-2 days, the solutions were filtered, dialyzed against Nanopure™ water, and lyophilized. To resuspend the DNA oligomers, filtered 1× PBS buffer was added to the container and samples were vortexed. If samples cannot be completely dissolved, gentle heating (˜35° C.) is applied.

### Example 7

### General Method for the Synthesis of pacDNAs

Amine-terminated polyethylene glycol (NH₂-PEG, 10 kDa) was reacted with norbornenyl N-hydroxysuccinimide ester to yield the norbornenyl PEG based on a reported method (Lu, X. et al., Providing Oligonucleotides with Steric Selectivity by Brush-Polymer-Assisted Compaction, J. Am. Chem. Soc., 137, 12466-12469 (2015); incorporated herein in its entirety). Norbornenyl PEG (200 mg/mL) and norbornenyl protDNA (100-300 nmol in 400 μL of DCM) were separately dissolved in anhydrous DCM under inert atmosphere. The vial containing norbornenyl PEG (30 eq.) was cooled to −20° C. using an ice-salt water bath, to which a DCM solution of 3^{rd}-generation Grubbs' catalyst (1 eq.) was added via a microsyringe. The reaction mixture was allowed to stir for 4 h at −20° C., and a solution of norbornenyl protDNA monomer (2 eq.) was added. The reaction mixture was further stirred overnight at room temperature. To terminate the polymerization, 150 μL of EVE was added to the reaction mixture and the solution was allowed to stir for another 2 h. After removing the volatile components under reduced pressure, the crude product was deprotected with methanolic ammonia, dried under reduced pressure, resuspended in 1 mL of water, and subjected to aqueous GPC. The fractions containing pacDNA were collected and dialyzed against Nanopure™ water to remove inorganic salt residues. The final solution was lyophilized to obtain pacDNAs as a yellow powder.

### Example 8

### General Method for the Synthesis of Amphiphilic DNA Diblock Copolymers

The hydrophobic monomers (2 and 3), protDNA monomer, and the 3^{rd}-generation Grubbs' catalyst were separately dissolved in anhydrous DCM under inert atmosphere. To the hydrophobic monomer solution was added the catalyst at a monomer:catalyst molar ratio of 10:1, and the reaction mixture was allowed to stir for 2 h at room temperature. Thereafter, an aliquot of the solution was then transferred to the protDNA solution with a catalyst:protDNA molar ratio of 10:1. The large excess of active polymer chain to protDNA monomer statistically ensures one DNA strand per polymer. The reaction mixture was further stirred for 4 h at room temperature, before EVE was added to terminate the polymerization. The crude product was deprotected by treatment with 500 μL of methanolic ammonia solution. After 4 hours of stirring, 3 mL of water was added to the mixture to precipitate unreacted hydrophobic polymers. The suspension was centrifuged, and the clear supernatant was collected, washed twice using 2 mL of DCM, passed through 0.45 μm nylon filter, and subjected to aqueous GPC to remove unreacted DNA. The fractions containing the diblock copolymers were collected and dialyzed against Nanopure™ water to remove inorganic residues. The final solution was lyophilized to yield dried product as a yellow powder.

### Example 9

### Hybridization Kinetics Assay

Fluorescein-labeled DNA samples were each dissolved in microcentrifuge tubes with 1× PBS (pH=7.4) to give final concentrations of 20 μM. Each solution (10 μL) was transferred to a quartz cuvette containing 980 μL of PBS buffer. The starting fluorescence of the mixture (excitation: 490 nm, emission: 520 nm) was recorded for 3 min. Thereafter, 10 μL of PBS solution containing complementary dabcyl-modified DNA strand (20 μM, 1.0 eq.) was added and the solution was rapidly and thoroughly mixed. The fluorescence was monitored every 3 sec using a Cary Eclipse fluorescence spectrometer, and monitoring was continued for 60 min. Endpoints were determined by adding an excess of the dabcyl-modified sequence followed by overnight incubation. The kinetic plots were normalized by treating starting points as 0% hybridization and endpoints as 100% hybridization. The measurements were taken in triplicates.

To investigate whether the hybridization and dehybridization properties of DNA were influenced by the brush architecture, a fluorescence quenching assay was adopted, where a quencher (dabcyl)-modified complementary strand was used to hybridize with the fluorescein-labeled DNA brush. Hybridization and thermal melting resulted in decreases and increases of the fluorescence, respectively, and the rates of changes reflect their kinetics. The hybridization kinetics of DNA brushes was initially as fast as that of the free DNA but slowed down slightly as the reaction proceeded, taking longer to reach complete duplex formation, and the trend is more obvious for brushes with larger DPs (FIG. 2g). The reduction in hybridization rate towards the end of the reaction may be attributed to the accumulation of negative charges on the nanostructure, which hinders subsequent reaction. On the other hand, thermal stability of DNA duplex in the brush form increased by ˜3° C. compared with free duplex, and with sharper melting transitions (FIG. 2h). A similar trend has been found with gold-cored SNAs, which can be explained by the elevated local salt concentration associated with densely arranged DNA. The increased salt concentration entropically stabilizes duplex DNA by masking their repulsive negative charges, resulting in higher melting temperatures. When a certain duplex within the nanostructure dehybridizes, salt concentration surrounding the duplex is reduced, making neighboring duplexes more prone to dissociate. These interpretations are applicable to the bottlebrush DNA.

### Example 10

### Monodisperse Oligomers and Cellular Uptake

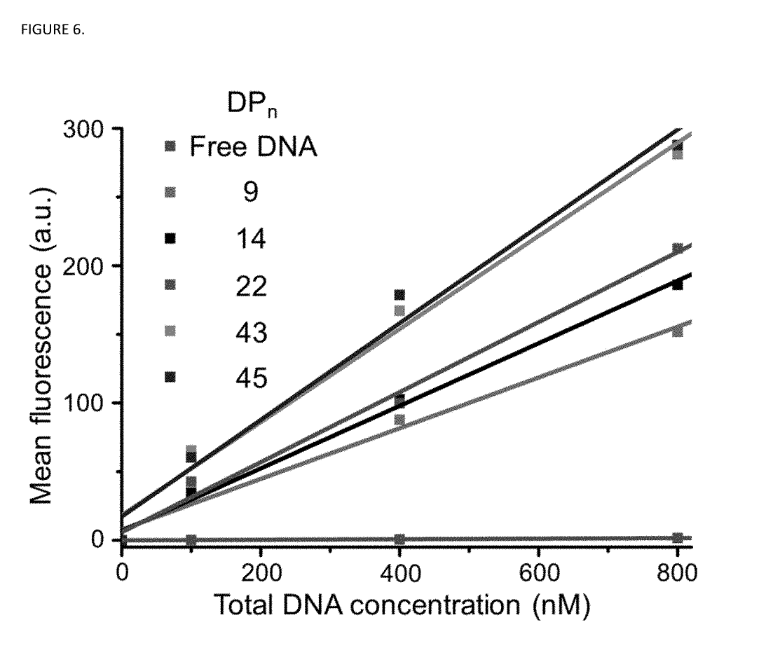

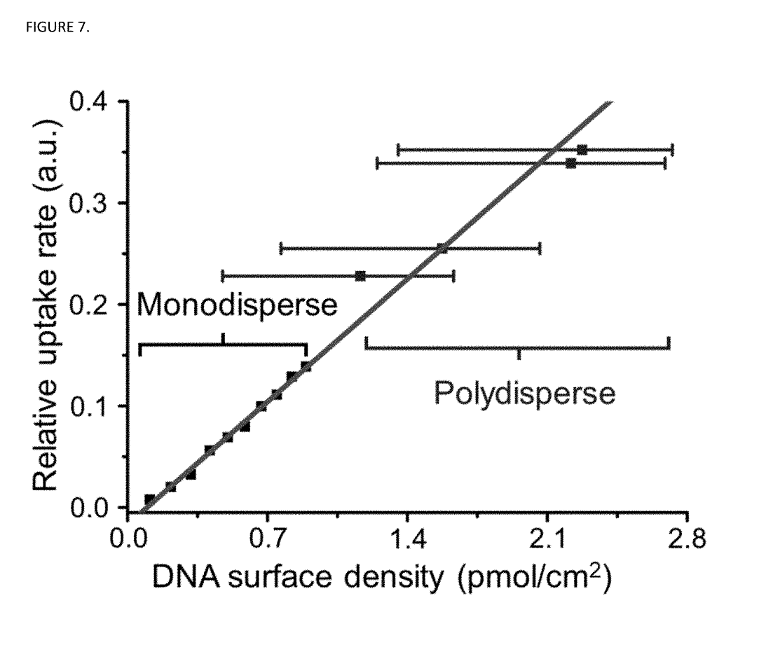

In a single ROMP reaction of protDNA (carried out at 2:1 or 3:1 monomer:catalyst ratio), oligomeric DNA with specific numbers of strands, including all odd-numbered ones, were simultaneously synthesized, and PAGE was used to isolate monodispersed fractions containing unimers through decamers (limited by gel resolution). On the size-density map for SNAs reported thus far, these molecular, monodisperse entities occupy the DNA density range (0.1-0.9 pmol/cm²) below that of typical SNAs, while the polydisperse brushes of higher DPs extend into the density range of SNAs (1.1-2.3 pmol/cm²). Thus, with identical chemical compositions, these well-defined structures enable an analysis of the effect of DNA surface density on cellular uptake. Human ovarian carcinoma cells (SKOV-3) were treated with varying concentrations (up to 800 nM of DNA) of fluorescein-labeled monodispersed oligomers as well as polydispersed brushes (to access higher densities) for 4 h, and cellular uptake was studied by confocal imaging and flow cytometry (total cell counts: 10,000). Confocal microscopy showed increasing cell-associated fluorescence (mainly from endosomal compartments) with increasing brush DPₙ. Flow cytometry showed that the uptake increased linearly with concentration, suggesting that saturation is far from being reached (FIG. 6 and Table 4). The extent of initial uptake for the brushes, as determined by the slope of the linear fit, is 90-170× greater than that of free DNA and increases with increasing DPs. Strikingly, if the slopes are plotted, which are indicative of the relative rate of cellular uptake, as a function of DNA surface density, another linear relationship comes into view (FIG. 7). The relative rate of uptake depends linearly with minimum variation (R²=0.994) on the DNA surface density in the monodisperse oligomer range, and the linear fit predicts the uptake of polydisperse brushes reasonably well. These results indicate that there is no transition or threshold density for a structure to behave like an SNA with regard to cell uptake. The uptake instead increases cumulatively with increasing DNA surface density, starting as early as a dimer.

TABLE 4

| | Mean fluorescence (a.u.) | | | |
|---|---|---|---|---|
| Sample | 0 nM | 100 nM | 400 nM | 800 nM |
| unimer | 0 | 1.51 | 4.12 | 6.45 |
| dimer | 0 | 3.86 | 13.09 | 16.22 |
| trimer | 0 | 10.91 | 25.46 | 27.58 |
| tetramer | 0 | 12.25 | 39.69 | 45.31 |
| pentamer | 0 | 12.81 | 41.89 | 55.81 |
| hexamer | 0 | 11.76 | 45.35 | 63.38 |
| heptamer | 0 | 12.57 | 52.25 | 78.98 |
| octamer | 0 | 12.48 | 59.61 | 87.12 |
| nonamer | 0 | 14.88 | 68.11 | 101.34 |
| decamer | 0 | 14.83 | 65.74 | 109.82 |
| brush DPₙ = 9 | 0 | 31.51 | 87.83 | 151.76 |
| brush DPₙ = 14 | 0 | 34.59 | 102.64 | 186.07 |
| brush DPₙ = 22 | 0 | 42.66 | 99.87 | 212.56 |
| brush DPₙ = 43 | 0 | 65.38 | 167.2 | 281.18 |
| brush DPₙ = 45 | 0 | 60.4 | 178.76 | 287.82 |

### Example 11

### Synthesis of DNA-PEG Diblock Brush Polymers

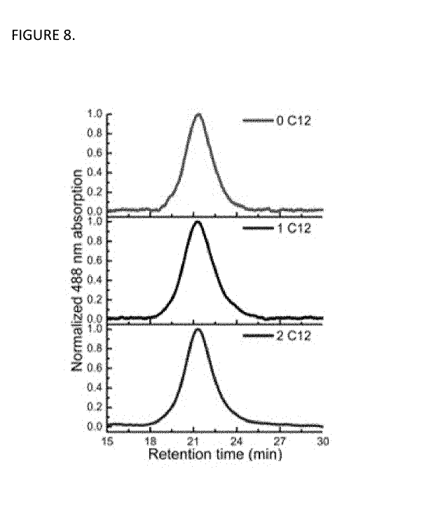

Norbornenyl PEG (10 kDa) was polymerized as the first block using 3^{rd}-generation Grubbs' catalyst with a 30:1 monomer:catalyst molar ratio at −20° C. in DCM. Upon completion of the polymerization of the first block (monitored by GPC), protDNA monomers were introduced to the reaction mixture at a monomer:catalyst ratio of 2:1 (monomer:catalyst). Three protDNA monomers with zero to two C12 spacers were used to test the effect of linker length on the incorporation yield of protDNA. After deprotection with methanolic ammonia, aqueous GPC showed ˜45%, 70%, and 70% incorporation for the protDNA with zero, one, and two C12 linkers, indicating that the steric hindrance of protDNA during ROMP can be circumvented by lengthening the spacing between norbornene and the protDNA. All three pacDNAs exhibited similar molecular weight (Mn ˜250 kDa by aqueous GPC; FIGS. 5b and 8), number-average hydrodynamic diameter (˜20.0±6.0 nm), potential (˜−4.0±0.6 mV) (FIG. 5d), and a spherical morphology as evidenced by TEM (FIG. 5c).

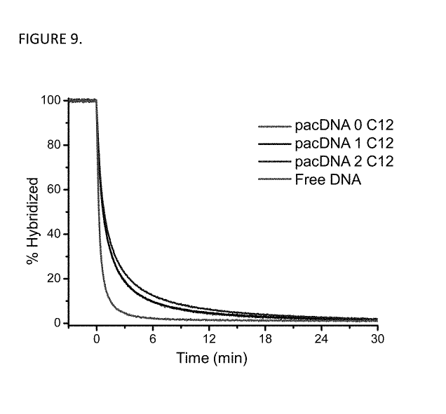

Hybridization of the one-pot pacDNA with a dabcyl-labeled complementary strand (fluorescence quenching assay) showed immediate and rapid hybridization, similar to that of free DNA (FIG. 9). To examine the inhibition of protein access, pre-formed free DNA and pacDNA duplexes were treated with DNase I (an endonuclease that non-specifically cleaves dsDNA). Upon degradation, the fluorophore-quencher pair is separated, leading to an increase of fluorescent signal, the rate of which is indicative of the degradation kinetics. It was found that all pacDNAs exhibited enhanced enzymatic stability compared with free DNA (2.7-7.8× longer half-life), and stability increased with decreasing numbers of the C12 linker, which suggests a depth-effect with respect to the brush backbone in steric protection (FIG. 5e), which has been previously observed. (Jia et al., Depth-profiling the Nuclease Stability and the Gene Silencing Efficacy of Brush-Architectured Poly(ethylene glycol)-DNA Conjugates, J. Am. Chem. Soc., 139, 10605-10608 (2017)). These results indicate that the one-pot pacDNA exhibits the same hallmark features of traditional pacDNAs obtained via multi-step syntheses.

What is claimed:

1. A method of synthesizing a protected norbornenyl DNA monomer or protected norbornenyl RNA monomer (protDNA) comprising the steps of:
1) providing a controlled pore glass (CPG) solid support for oligonucleotide synthesis, wherein the CPG solid support has covalently bound thereon a disulfide linker;
2) conducting a solid-phase oligonucleotide synthesis reaction with ultramild cyanoethyl (CE) phosphoramidites on the CPG solid support to form a protected oligonucleotide having a predetermined nucleotide sequence covalently bound at the 3′ end thereof to the disulfide linker;
2) cleaving the disulfide linker under conditions that release the protected oligonucleotide from the solid support, thereby forming a sulfhydryl group at the 3′end of the protected oligonucleotide; and
3) coupling an iodoacetyl- or a maleimide-functionalized norbornene monomer to the sulfhydryl group of the protected oligonucleotide via a SN2 or Michael addition reaction, respectively, to produce a protected norbornenyl DNA monomer or protected norbornenyl RNA monomer.

2. The method of claim 1 wherein at least one aliphatic linker with a length of from 9 to 18 atoms is incorporated at the 3′ end of the protected oligonucleotide.

3. The method of claim 1 wherein the ultramild cyanoethyl (CE) phosphoramidites are selected from the group consisting of Pac-dA-CE Phosphoramidite, Ac-dC-CE Phosphoramidite, iPr-Pac-dG-CE Phosphoramidite, and dT-CE Phosphoramidite.

4. A protected norbornenyl DNA monomer or protected norbornenyl RNA monomer (protDNA) made by the process of claim 1.

5. A method of synthesizing a DNA bottlebrush homopolymer comprising the steps of:
1) conducting ring opening metathesis polymerization (ROMP) of a plurality of the protected norbornenyl DNA monomer of claim 4 in the presence of an organic solvent and a heavy metal catalyst at a monomer:catalyst ratio of from 2:1 to 200:1 to form a DNA bottlebrush comprising a desired nucleotide sequence;
2) removing the catalyst; and
3) deprotecting the DNA bottlebrush with a deprotecting agent.

6. The method of claim 5, wherein the heavy metal catalyst is a 3^{rd} generation Grubbs' catalyst.

7. The method of claim 5, wherein the organic solvent is dichloromethane.

8. The method of claim 5, wherein deprotection is conducted with methanolic ammonia.

9. The method of claim 5, wherein deprotecting is conducted in a solution of K₂CO₃ in methanol, or a solution mixture of t-butylamine, methanol, and water, or a solution of concentrated ammonia.

10. The method of claim 9, wherein deprotection results in partial cleavage of the DNA bottlebrush.

11. The method of claim 5 wherein the monomer:catalyst ratio is from 5:1 to 80:1.

11. A DNA bottlebrush homopolymer made by the process of claim 5.

12. A method of synthesizing pacDNA comprising
1) reacting amine-terminated noncationic biocompatible monomers selected with norbornenyl N-hydroxysuccinimide ester to provide norbornenyl noncationic monomers;
2) dissolving the norbornenyl noncationic monomers in an organic solvent to form a first solution;
3) dissolving a plurality of the protected norbornenyl DNA monomer (protDNA) of claim 4 in an organic solvent to form a second solution;
4) forming a ROMP reaction mixture by adding a heavy metal catalyst to the first solution at a monomer:catalyst molar ratio of 10:1 to 100:1 and polymerizing the norbornenyl noncationic monomers in the ROMP reaction mixture to form a first block polymer having a predetermined sequence;
5) adding the second solution to the ROMP reaction mixture to provide a monomer:catalyst ratio of 1:1 to 10:1 and polymerizing the protDNA to form a pacDNA of predetermined sequence;
6) removing the catalyst; and
7) adding a deprotecting agent to remove the protecting groups.

13. The method of claim 12, wherein the heavy metal catalyst is a 3^{rd} generation Grubbs' catalyst.

14. The method of claim 12, wherein the noncationic biocompatible monomers are polyethylene glycol (PEG).

15. A pacDNA made by the process of claim 12.

16. A method of synthesizing DNA amphiphiles comprising:
1) reacting one or more hydrophobic, non-polar norbornene-protected monomers via ROMP using a monomer:catalyst ratio of from 3:1 to 50:1 until the monomers are substantially completely reacted to form a hydrophobic polymer;
2) adding a sub-stoichiometric amount of the protDNA of claim 4;
3) removing the catalyst;
4) adding a deprotecting agent to deprotect the protDNA; and
5) removing unreacted polymers or monomers to thereby isolate the DNA amphiphiles.

17. The method of claim 16, wherein the two hydrophobic, non-polar norbornenyl monomers are different from one another.

18. The method of claim 16, wherein a majority of the DNA amphiphiles comprises a single DNA strand.

19. The method of claim 16, wherein the deprotecting agent is methanolic ammonia.

20. The method of claim 12, wherein the deprotecting agent is methanolic ammonia.
